(19) United States
(12) Patent Application Publication
(10) Pub. No.: US 2023/0348420 A1
(43) Pub. Date: Nov. 2, 2023

(54) PROCESS FOR THE MANUFACTURE OF 1,4-DISUBSTITUTED PYRIDAZINE COMPOUNDS

(71) Applicant: Novartis AG, Basel (CH)

(72) Inventors: Nicolas AMIOT, Walheim (FR); Darija DEDIC, Basel (CH); Peng FU, Suzhou (CN); Fabrice GALLOU, Binningen (CH); Xingxian GU, Suzhou (CN); Cornelius HARLACHER, Reinach (CH); Siqian LIU, Suzhou (CN); Shuping YAO, Suzhou (CN); Jiong YE, Suzhou (CN); Jianguang ZHOU, Suzhou (CN)

(21) Appl. No.: 18/002,291

(22) PCT Filed: Jun. 24, 2021
(86) PCT No.: PCT/IB2021/055593

§ 371 Date: Dec. 19, 2022

(30) Foreign Application Priority Data
Jun. 25, 2020 (WO) PCT/CN2020/098302
Mar. 30, 2021 (WO) PCT/CN2021/083995

(51) Int. Cl. C07D 401/12; C07D 401/14; C07D 237/08; C07D 231/14

(52) U.S. Cl. CPC C07D 401/12; C07D 401/14; C07D 237/08; C07D 231/14

(57) ABSTRACT

The invention relates to a novel process, novel process step(s) and novel intermediate(s) useful for the preparation of 1,4-disubstituted pyridazine compounds, such as 5-(1H-Pyrazol-4-yl)-2-(6-((2,2,6,6-tetramethylpiperidin-4-yl)oxy)pyridazin-3-yl)phenol.

### FIELD OF THE INVENTION

The invention relates to a novel process, novel process step(s) and novel intermediate(s) useful for the preparation of 1,4-disubstituted pyridazine compounds, such as 5-(1H-Pyrazol-4-yl)-2-(6-((2,2,6,6-tetramethylpiperidin-4-yl)oxy)pyridazin-3-yl)phenol.

### BACKGROUND OF THE INVENTION

The present invention relates to processes for the preparation of 1,4-disubstituted pyridazine compounds, such as 5-(1H-Pyrazol-4-yl)-2-(6-((2,2,6,6-tetramethylpiperidin-4-yl)oxy)pyridazin-3-yl)phenol [i.e. Compound of formula (I) herein, also named branaplam].

5-(1H-Pyrazol-4-yl)-2-(6-((2,2,6,6-tetramethylpiperidin-4-yl)oxy)pyridazin-3-yl)phenol is a SMN (Survival of Motor Neuron) modulator useful e.g. for the treatment of SMA (Spinal Muscular Atrophy) and it has the structure (1):

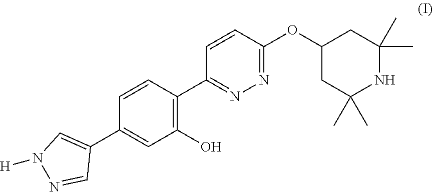 [SMILES: CC1(C)CC(Oc2ccc(-c3ccc(-c4cn[nH]c4)cc3O)nn2)CC(C)(C)N1]

5-(1H-Pyrazol-4-yl)-2-(6-((2,2,6,6-tetramethylpiperidin-4-yl)oxy)pyridazin-3-yl)phenol is described in WO2014/028459, in particular in Example 17-13 therein.

The synthesis of 5-(1H-Pyrazol-4-yl)-2-(6-((2,2,6,6-tetramethylpiperidin-4-yl)oxy)pyridazin-3-yl)phenol, as described in WO2014/028459, is summarized here below in Scheme 1. This synthesis utilizes sequential Suzuki cross-couplings to form intermediates 4a and (IIIa). The use of a protected phenol, as —OMe, boronic species of formula 3aa requires a late a deprotection step, which proved challenging on large scale.

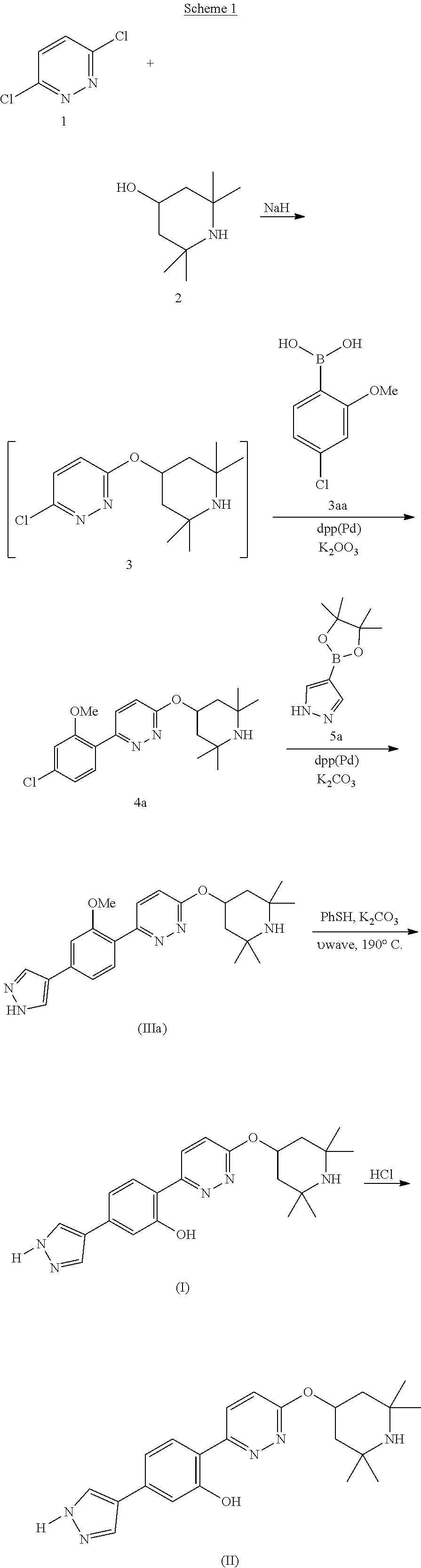

To avoid the late stage demethylation (i.e. deprotection) step, an alternative synthesis was developed. The alternative approach used instead 5-chloro-2-(4,4,5,5-tetramethyl-1,3,2-dioxaborolan-2-yl)phenol (herein named compound 3a) and it is published in J. Med. Chem. 2018, 61, 11021-11036. This alternative synthetic approach suffers from the potential intrinsic risk of producing dioxin impurities (“Health Assessment Document for Polychlorinated Dibenzo-p-dioxins”; U.S. Environmental Protection Agency. U.S. Government Printing Office: Washington, DC, 1985; EPA 600/8-84-014F.; “Estimating Exposure to Dioxin-Like Compounds, Vol. I: Executive Summary”, external review draft; Office of Health and Environmental Assessment Office of Research and Development. U.S. Government Printing Office: Washington, DC, June 1994; EPA/600/6-88/005Ca.; “Estimating Exposure to Dioxin-Like Compounds, Vol. II: Properties, Sources, Occurrence and Background Exposures”; external review draft; Office of Health and Environmental Assessment, Office of Research and Development. U.S. Government Printing Office: Washington, DC, June 1994; EPA/60/W6-88/005Cb.) derived from the polyhalogenated phenol intermediate 3b (i.e. precursor of compound 3a), and the risk of carrying such dioxin impurities into the final product.

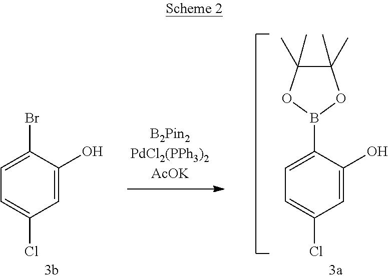 [SMILES: Oc1cc(Cl)ccc1Br ; CC1(C)OB(c2ccc(Cl)cc2O)OC1(C)C]

J. Org. Chem. 2018, 83, 2954-2958 also describes an alternative synthesis to compound of formula (I), which avoids the use of the phenol intermediate 3b, as it uses instead a protected phenol, as —OMe, which is later deprotected and further protected, as —OBn, thus making the whole synthesis economically not so attractive.

Therefore, there is a need to develop alternative efficient syntheses of compound of formula (I), which avoid both the use of the polyhalogenated phenol intermediate 3b, as well as the use of challenging (e.g. in a large scale) deprotecting steps.

### SUMMARY OF THE INVENTION

The invention relates to a novel process, novel process step(s) and novel intermediate(s) useful for the preparation of 1,4-disubstituted pyridazine compounds, such as 5-(1H-Pyrazol-4-yl)-2-(6-((2,2,6,6-tetramethylpiperidin-4-yl)oxy)pyridazin-3-yl)phenol, as described in Scheme 3 and Sections I to XI herein.

### BRIEF DESCRIPTION OF DRAWINGS

FIG. 1A: XRPD pattern of branaplam hydrochloride monohydrate (modification H_{B})

FIG. 1B: Crystal structure of branaplam hydrochloride monohydrate (modification H_{B})

### DETAILED DESCRIPTION OF THE INVENTION

The process, according to the present invention, for producing 1,4-disubstituted pyridazine compounds, such as compounds according to formula (I), or salt thereof, as defined herein, is summarized in Scheme 3.

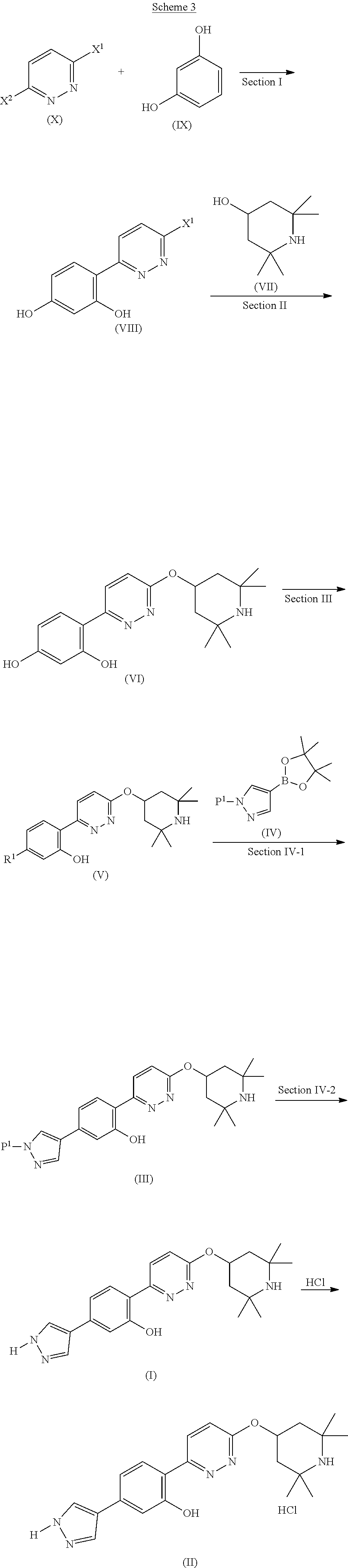

The compound of formula (III), or salt thereof, may be converted into the compound formula (I), or salt thereof, such as the HCl salt of formula (II), for example, as described in WO2014/028459, in particular as described in the relevant claims and examples, which are incorporated by reference herein.

Herein below, Sections 1l, Ill and IV, as such, are embodiments of the present invention. Furthermore, combination of two or more of (a), (b) and (c) above are also embodiments of the present invention.

### Section I: Preparation of a Compound of Formula (VIII) from a Compound of Formula (X)

A compound of formula (VIII), or salt thereof, in particular wherein X¹ is chloro, is prepared under Friedel-Crafts reaction conditions (e.g. in the Journal of Organic Chemistry 1990, 55(19), 5418-5420).

Embodiment 1: A method for preparing a compound of formula (VIII), or salt thereof,

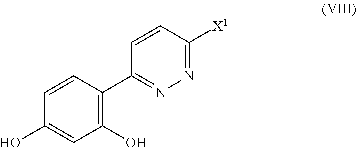

wherein
X¹ is halo;
comprising
reacting a compound of formula (X), or salt thereof,

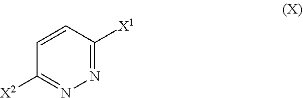

wherein
X¹ is halo;
X² is halo;
with a compound of formula (IX), or salt thereof,

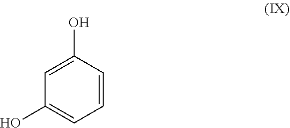

under Friedel-Crafts reaction conditions
to provide the compound of formula (VIII), or salt thereof.

Embodiment 1.1: A method for preparing a compound of formula (VIII), or salt thereof, according to embodiment 1, wherein X¹, for compounds of formula (X) and (VII), or in each instance a salt thereof, is chloro.

Embodiment 1.2: A method for preparing a compound of formula (VIII), or salt thereof, according to embodiment 1, wherein X², for compound of formula (X), or salt thereof, is chloro.

Embodiment 1.3: A method for preparing a compound of formula (VIII), or salt thereof, according to embodiment 1.1, wherein X², for compound of formula (X), or salt thereof, is chloro. In any one of embodiments 1, 1.1, 1.2 and 1.3, typically, Friedel-Craft reactions conditions are achieved in the presence of a Lewis acid. A Lewis acid is, for example, selected from the group consisting of AlBr₃, AlCl₃, GaCl₃, FeCl₃, SnCl₅, ZrCl₄, SnCl₄, BCl₃, BF₃, SbCl₃, Sc(OTf)₃ and Sm(OTf)₃.

Embodiment 1.4: A method for preparing a compound of formula (VIII), or salt thereof, according to any one of embodiments 1, 1.1, 1.2 and 1.3, wherein Friedel-Craft reactions conditions are achieved in the presence of AlCl₃.

### Section II: Preparation of a Compound of Formula (VI) from a Compound of Formula (VIII)

Embodiment 2: A method for preparing a compound of formula (VI), or salt thereof,

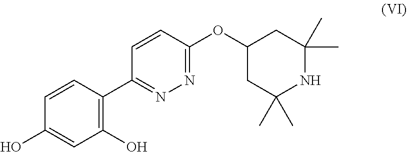 [SMILES: CC1(C)CC(Oc2ccc(-c3ccc(O)cc3O)nn2)CC(C)(C)N1]

comprising
reacting a compound of formula (VIII), or salt thereof,

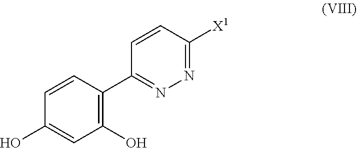

wherein
X¹ is halo;
with a compound of formula (VII), or salt thereof,

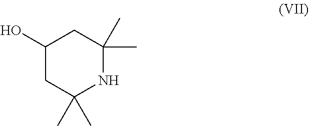

under nucleophilic aromatic substitution (SNAr) reaction conditions,
to provide the compound of formula (VI), or salt thereof.

Typically, nucleophilic aromatic substitution (SNAr) reaction conditions are achieved in the presence of a base. A base is, for example, an organic base or and inorganic base, for example, selected from the group consisting of ^{t}BuONa, ^{t}BuOLi, ^{t}BuOK, K₃PO₄, K₂CO₃. tetramethylguanidine, LDA and LHMDS, in particular ^{t}BuONa, ^{t}BuOLi, ^{t}BuOK, K₃PO₄ and K₂CO₃, such as ^{t}BuONa, ^{t}BuOLi and ^{t}BuOK.

Embodiment 2.1: A method for preparing a compound of formula (VI), or salt thereof, according to Embodiment 2, wherein X¹, for compound of formula (VIII), or salt thereof, is chloro.

Embodiment 2.2: A method for preparing a compound of formula (VI), or salt thereof, according to Embodiments 2 or 2.1, wherein nucleophilic aromatic substitution (SNAr) reaction conditions are achieved in the presence ^{t}BuONa.

Embodiment 2.3: A method for preparing a compound of formula (VI), or salt thereof, according to Embodiment 2.1, wherein nucleophilic aromatic substitution (SNAr) reaction conditions are achieved in the presence ^{t}BuONa.

### Section III: Preparation of a Compound of Formula (V) from a Compound of Formula (VI)

Embodiment 3: A method for preparing a compound of formula (V), or salt thereof,

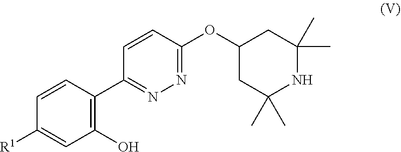

wherein R¹ is —OR², and R² is a hydroxyl activating group (e.g Ts)
comprising
reacting a compound of formula (VI), or salt thereof,

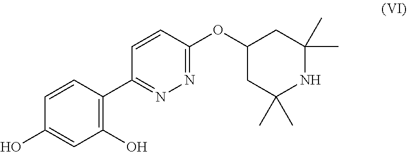 [SMILES: CC1(C)CC(Oc2ccc(-c3ccc(O)cc3O)nn2)CC(C)(C)N1]

under hydroxyl activating reaction conditions
to provide the compound of formula (V), or salt thereof.

For example, —OR² is —OTf or —OTs, thus R² is for example Tf or Ts.

Typically, hydroxyl activating reaction conditions are achieved, for example, with Tf₂O or TsCl.

Further hydroxyl activating reaction conditions are achieved, for example, with chlorodimethoxytriazine, dimethoxytriazine or morpholinium chloride.

In one embodiment, hydroxyl activating reaction conditions are achieved with TsCl, in the presence of a base, such an organic base (e.g. DBU or TMG) or an inorganic base (e.g. K₃PO₄ or K₂CO₃).

Embodiment 3.1: A method for preparing a compound of formula (V), or salt thereof, according to Embodiments 3, wherein hydroxyl activating reaction conditions are achieved in the presence of Tf₂O or TfCl to provide a compound of formula (V), or salt thereof, wherein R¹ is —OTf.

Embodiment 3.2: A method for preparing a compound of formula (V), or salt thereof, according to Embodiments 3, wherein hydroxyl activating reaction conditions are achieved in the presence of TsCl or Ts₂O to provide a compound of formula (V), or salt thereof, wherein R¹ is —OTs.

Embodiment 3.3: A method for preparing a compound of formula (V), or salt thereof, according to Embodiments 3.2, wherein hydroxyl activating reaction conditions are achieved in the presence of TsCl or Ts₂O and a base to provide a compound of formula (V), or salt thereof, wherein R¹ is —OTs.

Embodiment 3.4: A method for preparing a compound of formula (V), or salt thereof, according to Embodiments 3.3, wherein hydroxyl activating reaction conditions are achieved in the presence of TsCl and an inorganic base, for example K₃PO₄, to provide a compound of formula (V), or salt thereof, wherein R¹ is —OTs.

### Section IV-1: Preparation of a Compound of Formula (III) from a Compound of Formula (V)

### Embodiment 4

Embodiment 4a: A method for preparing a compound of formula (III), or salt thereof,

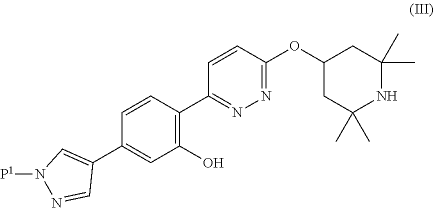

wherein
P¹ is a nitrogen protecting group,
comprising
reacting a compound of formula (V), or salt thereof,

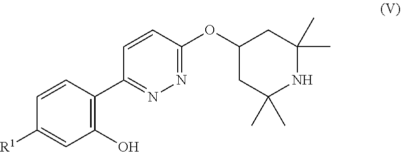

wherein
R¹ is —OR², and R² is a hydroxyl activating group (e.g Ts),
with a compound of formula (IV)

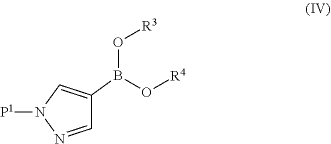

wherein
P¹ is a nitrogen protecting group, and
R³ is H or C₁₋₄alkyl;
R⁴ is H or C₁₋₄alkyl;
or R³ and R⁴ together to form the group

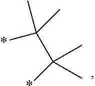 [SMILES: *C(C)(C)C(*)(C)C]

wherein the asterisk (*) denotes the point of attachment to each of the oxygen atoms attached to the boron atom,
under Suzuki coupling reaction conditions
to provide the compound of formula (III), or salt thereof.

Embodiment 4.1: A method for preparing a compound of formula (III-1), or salt thereof,

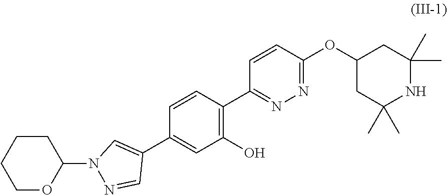 [SMILES: CC1(C)CC(Oc2ccc(-c3ccc(-c4cnn(C5CCCCO5)c4)cc3O)nn2)CC(C)(C)N1]

comprising
reacting a compound of formula (V), or salt thereof,

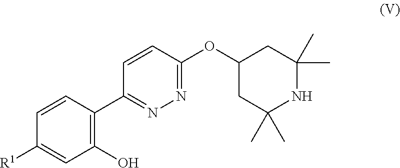

wherein R¹ is —OR², and R² is a hydroxyl activating group (e.g Ts)
with a compound of formula (IV-1)

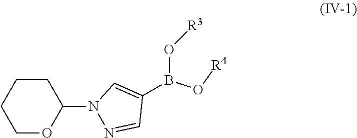

wherein
R³ is H or C₁₋₄alkyl;
R⁴ is H or C₁₋₄alkyl;
or R³ and R⁴ together to form the group

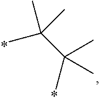

wherein the asterisk (*) denotes the point of attachment to each of the oxygen atoms attached to the boron atom,
under Suzuki coupling reaction conditions
to provide the compound of formula (III-1), or salt thereof.

Embodiment 4.2: A method for preparing a compound of formula (III), or salt thereof, according to Embodiment 4a or 4.1, wherein the Suzuki coupling reaction conditions are achieved with a palladium catalyst, for example [Pd(C₃H₅)|Cl]₂ in the presence of a phosphine ligand.

Embodiment 4.3: A method for preparing a compound of formula (III), or salt thereof, according to Embodiment 4.2, wherein the phosphine ligand is CyDPEPhos.

Embodiment 4.4: A method for preparing a compound of formula (III), or salt thereof, according to Embodiment 4.2 or 4.3, wherein Suzuki coupling reaction conditions are in the presence of base, such as an organic base or an inorganic base.

Embodiment 4.5: A method for preparing a compound of formula (III), or salt thereof, according to Embodiment 4.4, wherein the base is an inorganic base, for example, K₂CO₃, ^{t}BuOK, Cs₂CO₃, K₃PO₄ or NaOH.

Embodiment 4.6: A method for preparing a compound of formula (III), or salt thereof, according to Embodiment 4.4, wherein the base is an organic base, such as NEt₃.

Embodiment 4.7: A method for preparing a compound of formula (III), or salt thereof, according to any one of Embodiments 4a, 4.1, 4.2, 4.3, 4.4, 4.5 or 4.6, wherein R¹ is —OTs for compound of formula (V), or salt thereof.

Typically, Suzuki coupling reaction conditions are achieved with a palladium catalyst, for example in the presence of a base (e.g. in Smith, M., B.; March, J.; March's Advanced Organic Chemistry: Reactions, Mechanisms and Structure, 6th Edition, John Wiley & Sons, Inc., 2007; in particular as described in the relevant chapters thereof). Typical bases are, for example, inorganic bases (e.g. K₂CO₃, ^{t}BuOK, Cs₂CO₃, K₃PO₄, NaOH) and organic bases (e.g. NEt₃). Typical palladium catalysts are, for example, Pd(PPh₃)₄ or [Pd(C₃H₅)Cl]₂ in the presence of a phosphine ligand such as CyDPEPhos.

### Section IV-2: Preparation of a Compound of Formula (I) from a Compound of Formula (111)

Embodiment 4a-2: A method for preparing a compound of formula (I), or salt thereof,

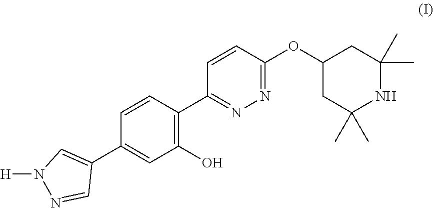 [SMILES: CC1(C)CC(Oc2ccc(-c3ccc(-c4cn[nH]c4)cc3O)nn2)CC(C)(C)N1]

comprising
reacting a compound of formula (III), or salt thereof,

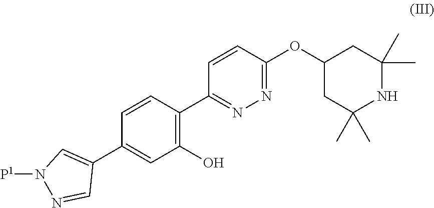

wherein
P¹ is a nitrogen protecting group,
under nitrogen deprotecting conditions to provide the compound of formula (I), or salt thereof.

In one embodiment nitrogen deprotecting conditions take place under acidic conditions, for example under inorganic acid conditions, such as with HCl.

In one embodiment, the method for preparing the compound of formula (I), or salt thereof, is optionally followed by crystallization.

Embodiment 4a-3: A method for preparing a compound of formula (I), or salt thereof,

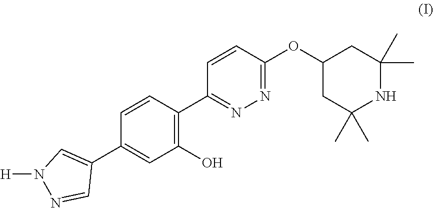 [SMILES: CC1(C)CC(Oc2ccc(-c3ccc(-c4cn[nH]c4)cc3O)nn2)CC(C)(C)N1]

comprising
reacting a compound of formula (Ill-1), or salt thereof,

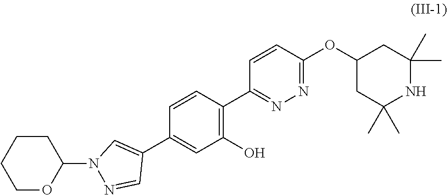

wherein
P¹ is a nitrogen protecting group
under nitrogen deprotecting conditions to provide the compound of formula (I), or salt thereof.

In one embodiment nitrogen deprotecting conditions take place under acidic conditions, for example under inorganic acid conditions, such as with HCl.

In one embodiment, the method for preparing the compound of formula (I), or salt thereof, is optionally followed by crystallization.

Embodiment 4a-4: A method for preparing a salt of the compound of formula (I),

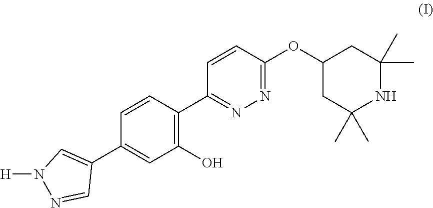 [SMILES: CC1(C)CC(Oc2ccc(-c3ccc(-c4cn[nH]c4)cc3O)nn2)CC(C)(C)N1]

comprising
reacting a compound of formula (III), or salt thereof,

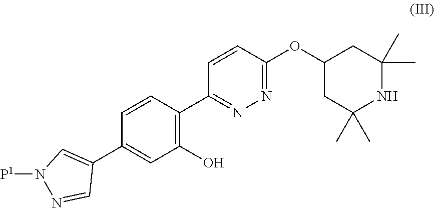

wherein
P¹ is a nitrogen protecting group,
under nitrogen deprotecting conditions to provide a salt of the compound of formula (I).

In one embodiment, nitrogen deprotecting conditions take place under acidic conditions, for example under inorganic acid conditions, such as with HCl, to provide a salt of the compound of formula (I),

In one embodiment, the method for preparing a salt of the compound of formula (I) is optionally followed by crystallization.

Embodiment 4a-5: A method for preparing a hydrochloride salt of the compound of formula (I),

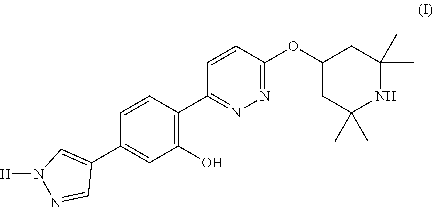 [SMILES: CC1(C)CC(Oc2ccc(-c3ccc(-c4cn[nH]c4)cc3O)nn2)CC(C)(C)N1]

comprising
reacting a compound of formula (III), or salt thereof,

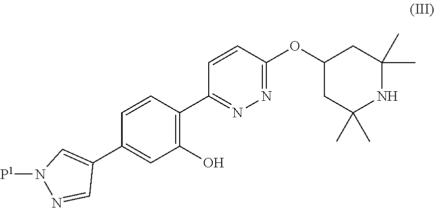

wherein
P¹ is a nitrogen protecting group,
with HCl to provide a hydrochloride salt of the compound of formula (I), such as the monohydrochloride monohydrate (i.e. 5-(1H-Pyrazol-4-yl)-2-{6-[(2,2,6,6-tetramethylpiperidin-4-yl)oxy]pyridazin-3-yl}phenol monohydrochloride monohydrate).

In one embodiment, the method for preparing a hydrochloride salt of the compound of formula (I) is optionally followed by crystallization.

Embodiment 4a-6: A method for preparing a salt of the compound of formula (I),

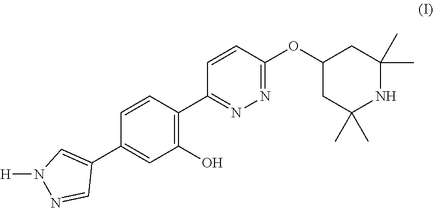 [SMILES: CC1(C)CC(Oc2ccc(-c3ccc(-c4cn[nH]c4)cc3O)nn2)CC(C)(C)N1]

comprising
reacting a compound of formula (III-1), or salt thereof,

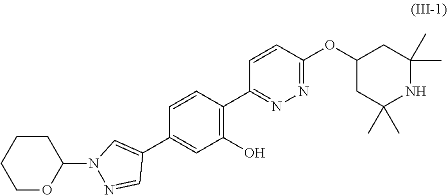

wherein
P¹ is a nitrogen protecting group
under nitrogen deprotecting conditions to provide a salt of the compound of formula (I).

In one embodiment nitrogen deprotecting conditions take place under acidic conditions, for example under inorganic acid conditions, such as with HCl.

In one embodiment, the method for preparing a salt of the compound of formula (I) is optionally followed by crystallization.

Embodiment 4a-7: A method for preparing a hydrochloride salt of the compound of formula (I),

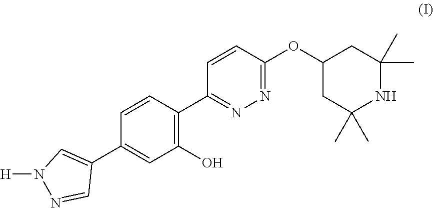 [SMILES: CC1(C)CC(Oc2ccc(-c3ccc(-c4cn[nH]c4)cc3O)nn2)CC(C)(C)N1]

comprising
reacting a compound of formula (III-1), or salt thereof,

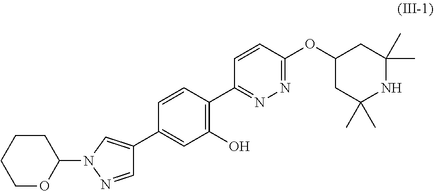

wherein
P¹ is a nitrogen protecting group
with HCl to provide a hydrochloride salt of the compound of formula (I), such as the monohydrochloride monohydrate (i.e. 5-(1H-Pyrazol-4-yl)-2-{6-[(2,2,6,6-tetramethylpiperidin-4-yl)oxy]pyridazin-3-yl}phenol monohydrochloride monohydrate).

In one embodiment, the method for preparing a hydrochloride salt of the compound of formula (I) is optionally followed by crystallization.

### Section Va: Two-Steps Preparation of a Compound of Formula (VI)

Embodiment 5a: A method for preparing a compound of formula (VI), or salt thereof,

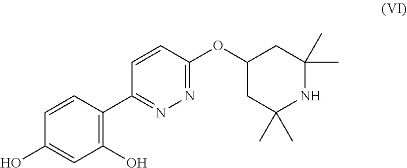 [SMILES: CC1(C)CC(Oc2ccc(-c3ccc(O)cc3O)nn2)CC(C)(C)N1]

comprising
   i) preparing a compound of formula (VIII), or salt thereof,

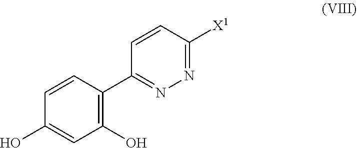

wherein
X¹ is halo, according to any one of methods of embodiments 1, 1.1, 1.2, 1.3 and 1,4; and
ii) reacting the compound of formula (VIII), or salt thereof, according to any one of methods of embodiments 2, 2.1, 2.2 and 2.3 to obtain the compound of formula (VI), or salt thereof.

### Section Vb: Two-Steps Preparation of a Compound of Formula (V)

Embodiment 5b: A method for preparing a compound of formula (V),

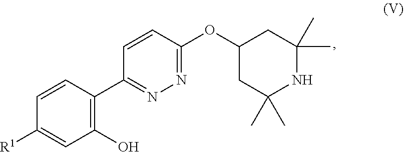

or salt thereof,
wherein R¹ is —OR², and R² is a hydroxyl activating group (e.g Ts), comprising:
   i) preparing a compound of formula (VI),

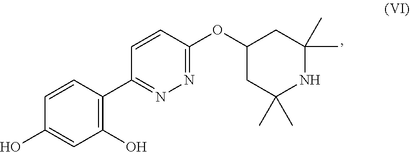 [SMILES: CC1(C)CC(Oc2ccc(-c3ccc(O)cc3O)nn2)CC(C)(C)N1]

or salt thereof,
according to any one of methods of embodiments 2, 2.1, 2.2 and 2.3; and
   ii) reacting the compound of formula (VI), or salt thereof, according to any one of methods of embodiments 3, 3.1, 3.2, 3.3 and 3.4, to obtain the compound of formula (V), or salt thereof.

Embodiment 5.1b: A method for preparing a compound of formula (V),

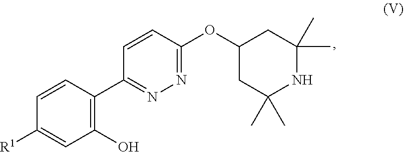

or salt thereof,
wherein R¹ is —OR², and R² is a hydroxyl activating group (e.g Ts),
comprising:
   i) preparing a compound of formula (VI),

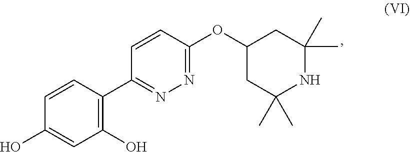 [SMILES: CC1(C)CC(Oc2ccc(-c3ccc(O)cc3O)nn2)CC(C)(C)N1]

or salt thereof,
according to method of embodiment 2.3; and
   ii) reacting the compound of formula (VI), or salt thereof, according to any one of methods of embodiments 3, 3.1, 3.2, 3.3 and 3.4, to obtain the compound of formula (V), or salt thereof.

### Section VI: Two-Steps Preparation of a Compound of Formula (III)

### Embodiment 6

Embodiments 6a: A method for preparing a compound of formula (III), or salt thereof,

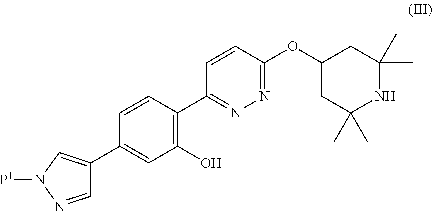

wherein P¹ is a nitrogen-protecting group
comprising:
   i) preparing a compound of formula (V),

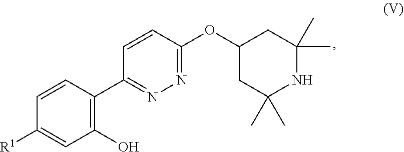

or salt thereof,
wherein R¹ is —OR², and R² is a hydroxyl activating group (e.g Ts),
from the compound of formula (VI),

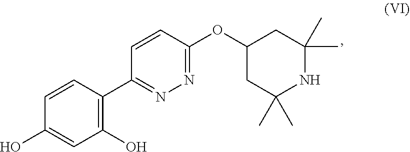 [SMILES: CC1(C)CC(Oc2ccc(-c3ccc(O)cc3O)nn2)CC(C)(C)N1]

or salt thereof,
according to any one of methods of embodiments 3, 3.1, 3.2, 3.3 and 3.4; and
   ii) reacting the compound of formula (V), or salt thereof, according to any one of methods of embodiments 4a, 4.1, 4.2, 4.3, 4.4, 4.5, 4.6 and 4.7, to obtain the compound of formula (III), or salt thereof.

Embodiment 6.1a: A method for preparing a compound of formula (III-1), or salt thereof,

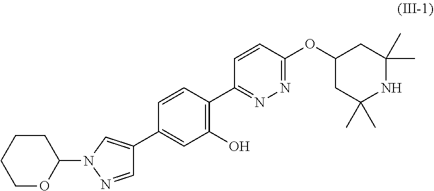 [SMILES: CC1(C)CC(Oc2ccc(-c3ccc(-c4cnn(C5CCCCO5)c4)cc3O)nn2)CC(C)(C)N1]

comprising:
   i) preparing a compound of formula (V),

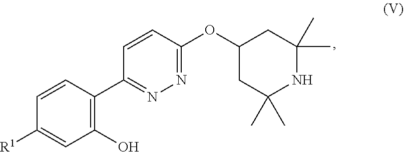

or salt thereof,
wherein R¹ is —OR², and R² is a hydroxyl activating group (e.g Ts),
from the compound of formula (VI),

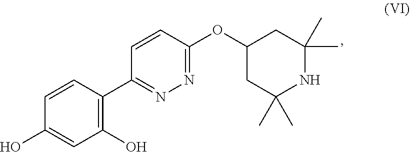 [SMILES: CC1(C)CC(Oc2ccc(-c3ccc(O)cc3O)nn2)CC(C)(C)N1]

or salt thereof,
according to method of embodiment 3.4; and
   ii) reacting the compound of formula (V), or salt thereof, according to any one of methods of embodiments 4a, 4.1, 4.2, 4.3, 4.4, 4.5, 4.6 and 4.7, to obtain the compound of formula (III), or salt thereof.

Embodiment 6b: A method for preparing a compound of formula (Ill-1), or salt thereof,

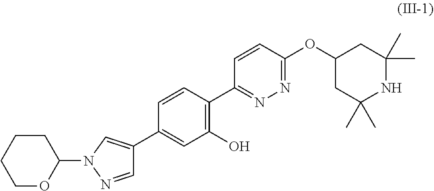 [SMILES: CC1(C)CC(Oc2ccc(-c3ccc(-c4cnn(C5CCCCO5)c4)cc3O)nn2)CC(C)(C)N1]

comprising:
   i) preparing a compound of formula (V),

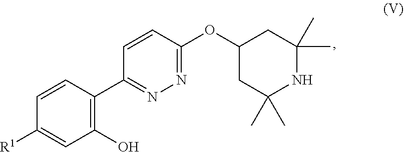

or salt thereof,
wherein R¹ is —OR², and R² is a hydroxyl activating group (e.g Ts),
from the compound of formula (VI),

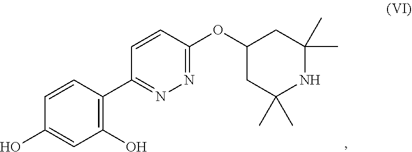 [SMILES: CC1(C)CC(Oc2ccc(-c3ccc(O)cc3O)nn2)CC(C)(C)N1]

or salt thereof,
according to any one of methods of embodiments 3, 3.1, 3.2, 3.3 and 3.4; and
   ii) reacting the compound of formula (V), or salt thereof, according to any one of methods of embodiments 4.1, 4.2, 4.3, 4.4, 4.5, 4.6 and 4.7, to obtain the compound of formula (III-1), or salt thereof.

Embodiment 6.1 b: A method for preparing a compound of formula (Ill-1), or salt thereof,

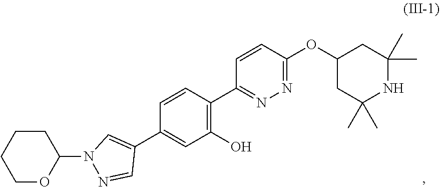 [SMILES: CC1(C)CC(Oc2ccc(-c3ccc(-c4cnn(C5CCCCO5)c4)cc3O)nn2)CC(C)(C)N1]

comprising:
   i) preparing a compound of formula (V),

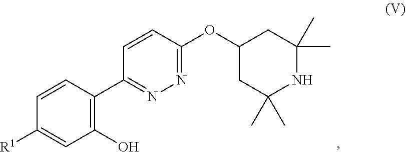

or salt thereof,
wherein R¹ is —OR², and R² is a hydroxyl activating group (e.g Ts),
from the compound of formula (VI),

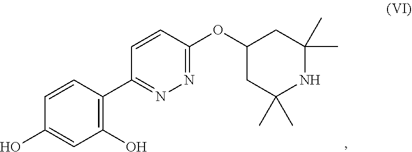 [SMILES: CC1(C)CC(Oc2ccc(-c3ccc(O)cc3O)nn2)CC(C)(C)N1]

or salt thereof,
according to method of embodiment 3.4; and
   ii) reacting the compound of formula (V), or salt thereof, according to any one of methods of embodiments 4.1, 4.2, 4.3, 4.4, 4.5, 4.6 and 4.7, to obtain the compound of formula (III), or salt thereof.

### Section VII: Three Steps Preparation of a Compound of Formula (III)

Embodiment 7: A method for preparing a compound of formula (II-1), or salt thereof,

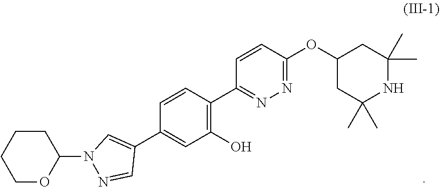 [SMILES: CC1(C)CC(Oc2ccc(-c3ccc(-c4cnn(C5CCCCO5)c4)cc3O)nn2)CC(C)(C)N1]

according to the method of embodiments 6b or 6.1b, wherein the compound of formula (VI) or salt thereof is prepared according to any one of methods of embodiments 2, 2.1, 2.2 and 2.3.

Embodiment 7.1: A method for preparing a compound of formula (Ill-1), or salt thereof,

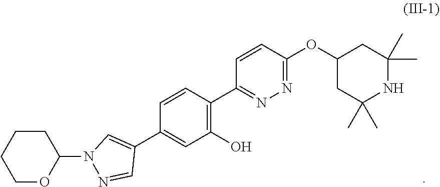 [SMILES: CC1(C)CC(Oc2ccc(-c3ccc(-c4cnn(C5CCCCO5)c4)cc3O)nn2)CC(C)(C)N1]

according to the method of embodiments 6b or 6.1b, wherein the compound of formula (VI) or salt thereof is prepared according to method of embodiment 2.3.

### Section VIII: Two or more steps preparation of a compound of formula (I)

### Embodiment 8.1

A method for preparing a compound of formula (I), or salt thereof,

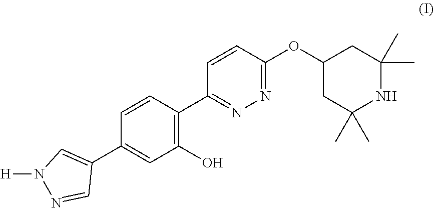 [SMILES: CC1(C)CC(Oc2ccc(-c3ccc(-c4cn[nH]c4)cc3O)nn2)CC(C)(C)N1]

comprising
   i) reacting a compound of formula (V), or salt thereof,

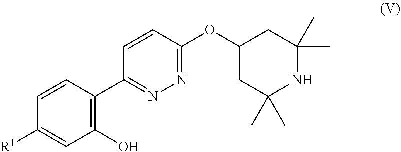

wherein
R¹ is —OR², and R² is a hydroxyl activating group (e.g. Ts),
according to any one of methods of embodiments 4a, 4.2, 4.3, 4.4, 4.5, 4.6 and 4.7,
to provide the compound of formula (III), or salt thereof,

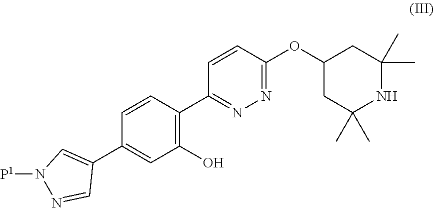

wherein
P¹ is a nitrogen protecting group; and
   ii) reacting the compound of formula (III), or salt thereof, according to the method of embodiment 4a-2 to provide the compound of formula (I), or salt thereof.

In one embodiment, the method for preparing the compound of formula (I), or salt thereof, is optionally followed by crystallization.

### Embodiment 8.2

A method for preparing a compound of formula (I), or salt thereof,

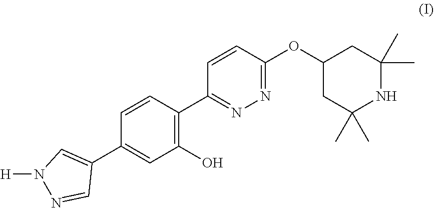 [SMILES: CC1(C)CC(Oc2ccc(-c3ccc(-c4cn[nH]c4)cc3O)nn2)CC(C)(C)N1]

comprising
   i) reacting a compound of formula (V), or salt thereof,

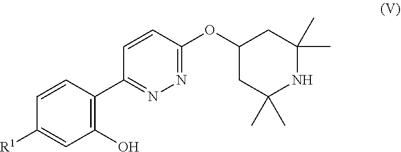

wherein
R¹ is —OR², and R² is a hydroxyl activating group (e.g. Ts),
according to the method of embodiment 4.1,
to provide the compound of formula (Ill-1), or salt thereof,

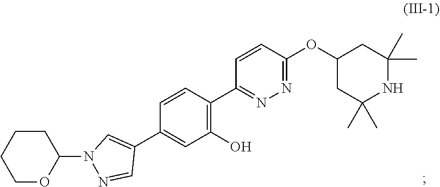 [SMILES: CC1(C)CC(Oc2ccc(-c3ccc(-c4cnn(C5CCCCO5)c4)cc3O)nn2)CC(C)(C)N1]

and
ii) reacting the compound of formula (Ill-1), or salt thereof, according to the method of embodiment 4a-3 to provide the compound of formula (I), or salt thereof.

In one embodiment, the method for preparing the compound of formula (I), or salt thereof, is optionally followed by crystallization.

Embodiment 8.3: A method for preparing a compound of formula (I), or salt thereof,

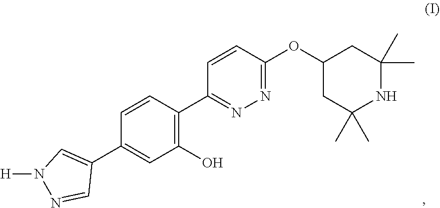 [SMILES: CC1(C)CC(Oc2ccc(-c3ccc(-c4cn[nH]c4)cc3O)nn2)CC(C)(C)N1]

according to the method of embodiments 8.1 or 8.2,
wherein the compound of formula (V), or salt thereof, is prepared according to any one of methods of embodiments 3, 3.1, 3.2, 3.3 and 3.4, in particular, is prepared according to the method of embodiment 3.4.

In one embodiment, the method for preparing the compound of formula (I), or salt thereof, is optionally followed by crystallization.

Embodiment 8.4: A method for preparing a compound of formula (V), or salt thereof,

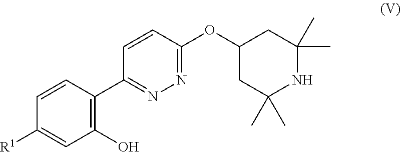

wherein
R¹ is —OR², and R² is a hydroxyl activating group (e.g. Ts),
according to the method of embodiments 5b or 5.1 b,
wherein the compound of formula (VIII), or salt thereof, is prepared according to any one of methods of embodiments 1, 1.1, 1.2, 1.3. and 1.4, in particular, is prepared according to the method of embodiment 1.4.

### Embodiment 8.5

A method for preparing a compound of formula (I), or salt thereof,

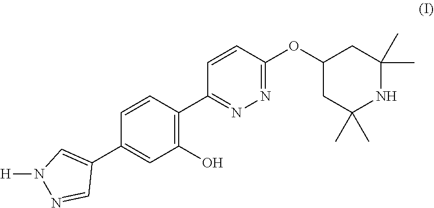 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof,
according to any one of methods of embodiments 3, 3.1, 3.2, 3.3 and 3.4, to provide the compound of formula (V), or salt thereof,
   ii) reacting the compound of formula (V), or salt thereof, according to any one of methods of embodiments 4a, 4.1, 4.2, 4.3, 4.4, 4.5, 4.6 and 4.7 to provide the compound of formula (III), or salt thereof; and
   iii) reacting the compound of formula (III), or salt thereof; according to the method of embodiments 4a-2 and 4a-3, to provide the compound of formula (I), or salt thereof.

In one embodiment, the method for preparing the compound of formula (I), or salt thereof, is optionally followed by crystallization.

### Embodiment 8.6

A method for preparing a compound of formula (V), or salt thereof,

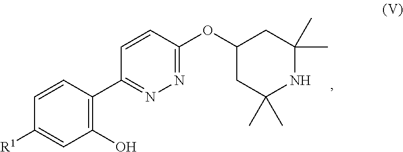

wherein R¹ is —OR², and R² is a hydroxyl activating group (e.g Ts)
comprising
   i) reacting the compound of formula (X), or salt thereof,
according to any one of methods of embodiments 1, 1.1, 1.2, 1.3 and 1.4, to provide the compound of formula (VIII), or salt thereof;
   ii) reacting the compound of formula (VIII), or salt thereof, according to any one of methods of embodiments 2, 2.1, 2.2 and 2.3 to provide the compound of formula (VI), or salt thereof; and
   iii) reacting the compound of formula (VI), or salt thereof; according to any one of methods of embodiments 3, 3.1, 3.2, 3.3 and 3.4, to provide the compound of formula (V), or salt thereof.

### Embodiment 8.7

A method for preparing a compound of formula (I), or salt thereof,

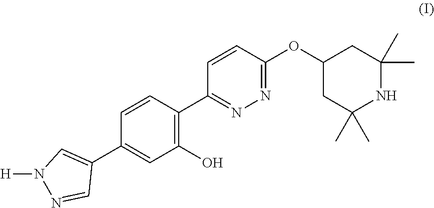 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof, according to any one of methods of embodiments 3, 3.1, 3.2, 3.3 and 3.4, to provide the compound of formula (V), or salt thereof,
   ii) reacting the compound of formula (V), or salt thereof, according to any one of methods of embodiments 4a, 4.1, 4.2, 4.3, 4.4, 4.5, 4.6 and 4.7 to provide the compound of formula (III), or salt thereof; and
   iii) reacting the compound of formula (III), or salt thereof; according to the method of embodiments 4a-2 and 4a-3, to provide the compound of formula (I), or salt thereof.

In one embodiment, the method for preparing the compound of formula (I), or salt thereof, is optionally followed by crystallization.

### Embodiment 8.8

A method for preparing a compound of formula (III), or salt thereof,

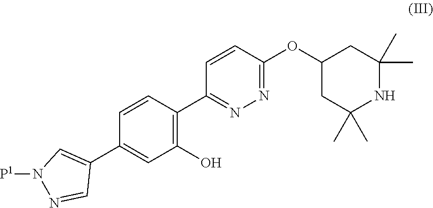

wherein
P¹ is a nitrogen protecting group,
comprising
   i) reacting the compound of formula (X), or salt thereof, according to any one of methods of embodiments 1, 1.1, 1.2, 1.3 and 1.4, to provide the compound of formula (VIII), or salt thereof;
   ii) reacting the compound of formula (VIII), or salt thereof, according to any one of methods of embodiments 2, 2.1, 2.2 and 2.3 to provide the compound of formula (VI), or salt thereof;
   iii) reacting the compound of formula (VI), or salt thereof; according to any one of methods of embodiments 3, 3.1, 3.2, 3.3 and 3.4, to provide a compound of formula (V), or salt thereof; and
   iv) reacting the compound of formula (V), or salt thereof; according to any one of methods of embodiments 4a, 4.1, 4.2, 4.3, 4.4, 4.5, 4.6 and 4.7, to provide the compound of formula (III), or salt thereof.

### Embodiment 8.9

A method for preparing a compound of formula (I), or salt thereof,

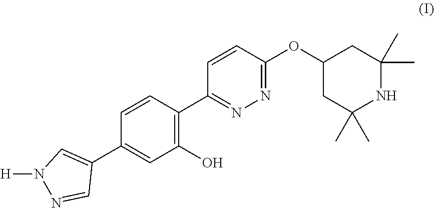 [SMILES: CC1(C)CC(Oc2ccc(-c3ccc(-c4cn[nH]c4)cc3O)nn2)CC(C)(C)N1]

comprising
   i) reacting the compound of formula (X), or salt thereof, according to any one of methods of embodiments 1, 1.1, 1.2, 1.3 and 1.4, to provide the compound of formula (VIII), or salt thereof;
   ii) reacting the compound of formula (VIII), or salt thereof, according to any one of methods of embodiments 2, 2.1, 2.2 and 2.3 to provide the compound of formula (VI), or salt thereof;
   iii) reacting the compound of formula (VI), or salt thereof; according to any one of methods of embodiments 3, 3.1, 3.2, 3.3 and 3.4, to provide a compound of formula (V), or salt thereof;
   iv) reacting the compound of formula (V), or salt thereof; according to any one of methods of embodiments 4a, 4.1, 4.2, 4.3, 4.4, 4.5, 4.6 and 4.7, to provide the compound of formula (III), or salt thereof; and
   v) reacting the compound of formula (III), or salt thereof, according to any one of methods of embodiments 4a-2 and 4a-3 to provide the compound of formula (I), or salt thereof.

In one embodiment, the method for preparing the compound of formula (I), or salt thereof, is optionally followed by crystallization.

### Embodiment 8.10

A method for preparing a compound of formula (I), or salt thereof,

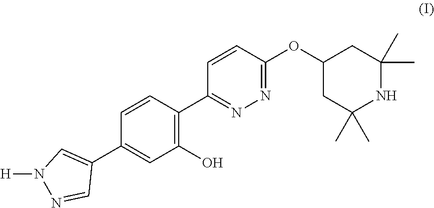 [SMILES: CC1(C)CC(Oc2ccc(-c3ccc(-c4cn[nH]c4)cc3O)nn2)CC(C)(C)N1]

comprising
   i) reacting the compound of formula (VIII), or salt thereof, according to any one of methods of embodiments 2, 2.1, 2.2 and 2.3 to provide the compound of formula (VI), or salt thereof;
   ii) reacting the compound of formula (VI), or salt thereof; according to any one of methods of embodiments 3, 3.1, 3.2, 3.3 and 3.4, to provide a compound of formula (V), or salt thereof;
   iii) reacting the compound of formula (V), or salt thereof; according to any one of methods of embodiments 4a, 4.1, 4.2, 4.3, 4.4, 4.5, 4.6 and 4.7, to provide the compound of formula (III), or salt thereof; and
   iv) reacting the compound of formula (III), or salt thereof, according to any one of methods of embodiments 4a-2 and 4a-3 to provide the compound of formula (I), or salt thereof.

In one embodiment, the method for preparing the compound of formula (I), or salt thereof, is optionally followed by crystallization.

### Section IX: Compounds of the Invention

The invention relates also to novel intermediates described herein, especially those leading to compounds mentioned as preferred herein, in particular:

Embodiment 9.1: A Compound of Formula (V)

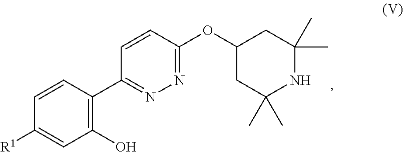

or salt thereof,
wherein R¹ is —OR², and R² is a hydroxyl activating group (e.g Ts).

Embodiment 9.2: A Compound of Formula (V)

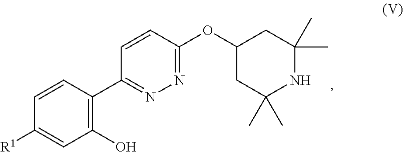

or salt thereof,
wherein R¹ is —OTs.

Embodiment 9.3: A Compound of Formula (VI),

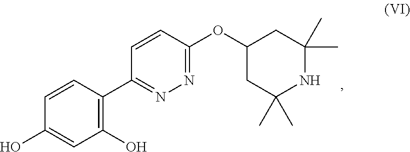 [SMILES: CC1(C)CC(Oc2ccc(-c3ccc(O)cc3O)nn2)CC(C)(C)N1]

or salt thereof.

Embodiment 9.4: A Compound of Formula (III-1),

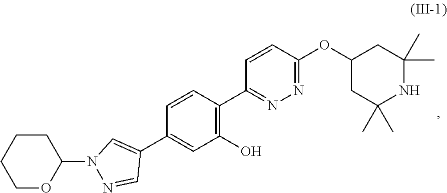 [SMILES: CC1(C)CC(Oc2ccc(-c3ccc(-c4cnn(C5CCCCO5)c4)cc3O)nn2)CC(C)(C)N1]

or a salt thereof.

Embodiment 9.5: A Compound of Formula (III),

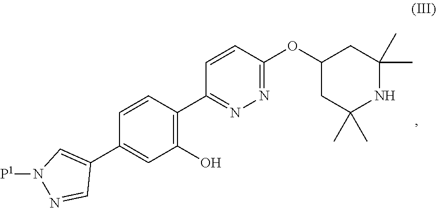

or a salt thereof,
wherein P¹ is a nitrogen protecting group.

Embodiment 9.6: A Compound of Formula (VIII), or Salt Thereof,

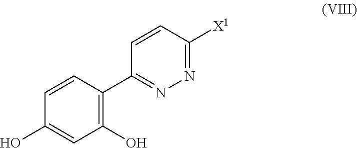

wherein
X¹ is halo (e.g Cl).

### Section IXa: Preparation of a Compound of Formula (IV)

### Embodiment 9a

A method for preparing a compound of formula (IV)

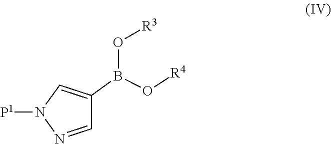

wherein
P¹ is a nitrogen protecting group
R³ is H or C₁₋₄alkyl;
R⁴ is H or C₁₋₄alkyl;
or R³ and R⁴ together to form the group

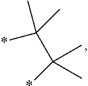 [SMILES: *C(C)(C)C(*)(C)C]

wherein * denotes the point of attachment to each of the oxygen atoms attached to the boron atom,
comprising
   i) reacting a compound of formula (XI), or salt thereof,

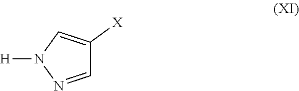

wherein X is halogen (e.g. iodo)
under nitrogen protecting conditions to provide the compound of formula (XII), or salt thereof

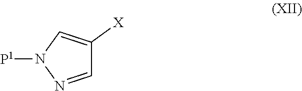

wherein X is halogen (e.g. iodo)
P¹ is a nitrogen protecting group;
   ii) reacting the compound of formula (XII), or salt thereof with a compound of formula (XIII),

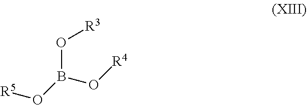

wherein
R³ is H or C₁₋₄alkyl;
R⁴ is H or C₁₋₄alkyl;
R⁵ is C₁₋₄alkyl;
or R³ and R⁴ together to form the group

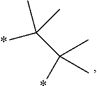 [SMILES: *C(C)(C)C(*)(C)C]

wherein * denotes the point of attachment to each of the oxygen atoms attached to the boron atom,
under Grignard reaction conditions
to provide the compound of formula (IV), or salt thereof.

As used herein the term “Grignard reaction conditions” comprises Grignard type reagents such as XMgR, wherein X is halo (e.g. Cl) and R is C₁₋₄alkyl, for example iPrMgCl.

In one embodiment nitrogen protecting conditions take place under acidic conditions, for example under organic acid conditions, such as with methanesulfonic acid. In one embodiment, nitrogen protecting conditions comprise reaction with 3,4-dihydro-2H-pyran under organic acid conditions, such as with methanesulfonic acid.

In one embodiment the compound of formula (IV) is of formula (IV-1)

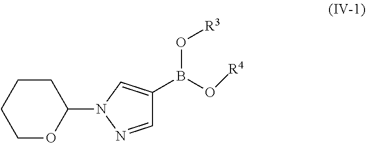

wherein
R³ is H or C₁₋₄alkyl;
R⁴ is H or C₁₋₄alkyl;
or R³ and R⁴ together to form the group

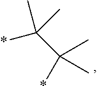 [SMILES: *C(C)(C)C(*)(C)C]

wherein the asterisk (*) denotes the point of attachment to each of the oxygen atoms attached to the boron atom.

### Section X: Further Embodiments

Embodiment 10.1: A method for preparing a compound of formula (VI), or salt thereof,

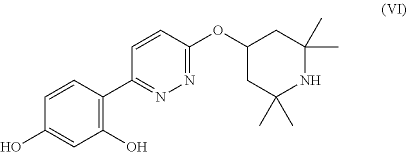 [SMILES: CC1(C)CC(Oc2ccc(-c3ccc(O)cc3O)nn2)CC(C)(C)N1]

comprising
reacting a compound of formula (VIII), or salt thereof,

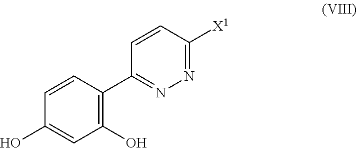

wherein
X¹ is halo;
with a compound of formula (VII), or salt thereof,

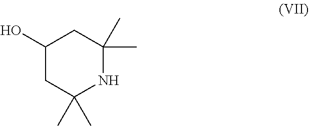

under nucleophilic aromatic substitution (SNAr) reaction conditions,
to provide the compound of formula (VI), or salt thereof.

Embodiment 10.2: The method for preparing a compound of formula (VI), or salt thereof, according to embodiment 10.1, wherein X¹, for compound of formula (VIII), or salt thereof, is chloro.

Embodiment 10.3: The method for preparing a compound of formula (VI), or salt thereof, according to embodiments 10.1 or 10.2, wherein nucleophilic aromatic substitution (SNAr) reaction conditions are achieved in the presence of a base, such an organic base, for example, ^{t}BuONa.

Embodiment 10.4: A method for preparing a compound of formula (V), or salt thereof,

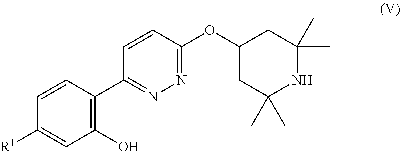

wherein R¹ is —OR², and R² is a hydroxyl activating group (e.g Ts)
comprising
reacting a compound of formula (VI), or salt thereof,

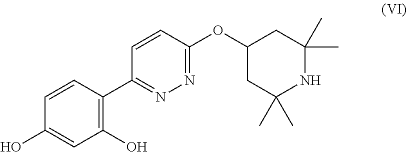 [SMILES: CC1(C)CC(Oc2ccc(-c3ccc(O)cc3O)nn2)CC(C)(C)N1]

under hydroxyl activating reaction conditions
to provide the compound of formula (V), or salt thereof.

Embodiment 10.5: A method for preparing a compound of formula (V), or salt thereof, according to embodiment 10.4, wherein hydroxyl activating reaction conditions are achieved in the presence of Tf₂O to provide a compound of formula (V), or salt thereof, wherein R¹ is —OTf.

Embodiment 10.6: A method for preparing a compound of formula (V), or salt thereof, according to embodiment 10.4, wherein hydroxyl activating reaction conditions are achieved in the presence of TsCl to provide a compound of formula (V), or salt thereof, wherein R¹ is —OTs.

Embodiment 10.7: The method for preparing a compound of formula (V), or salt thereof, according to embodiment 10.6, wherein hydroxyl activating reaction conditions are achieved in the presence of TsCl and a base, such as an inorganic base, for example K₃PO₄, to provide a compound of formula (V), or salt thereof, wherein R¹ is —OTs.

Embodiment 10.8: A method for preparing a compound of formula (III), or salt thereof,

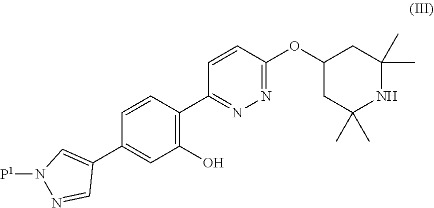

wherein P¹ is a nitrogen protecting group
comprising
reacting a compound of formula (V), or salt thereof,

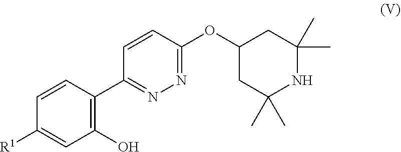

wherein R¹ is —OR², and R² is a hydroxyl activating group (e.g Ts)
with a compound of formula (IV)

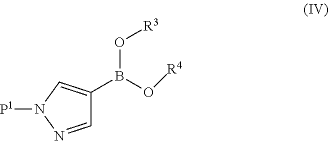

wherein
P¹ is a nitrogen protecting group
R³ is H or C₁₋₄alkyl;
R⁴ is H or C₁₋₄alkyl;
or R³ and R⁴ together to form the group

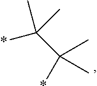 [SMILES: *C(C)(C)C(*)(C)C]

wherein * denotes the point of attachment to each of the oxygen atoms attached to the boron atom,
under Suzuki coupling reaction conditions
to provide the compound of formula (III), or salt thereof.

Embodiment 10.9: A method for preparing a compound of formula (III), or salt thereof, according to embodiment 10.8, wherein the compound of formula (IV) is

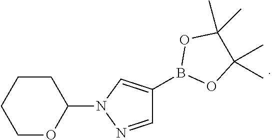 [SMILES: CC1(C)OB(c2cnn(C3CCCCO3)c2)OC1(C)C]

### Embodiment 10.10

The method for preparing a compound of formula (III), or salt thereof, according to embodiments 10.8 or 10.9, wherein the Suzuki coupling reaction conditions are achieved in the presence of [Pd(C₃H₅)|Cl]₂ in the presence of a phosphine ligand, such as CyDPEPhos.

Embodiment 10.11: The method for preparing a compound of formula (III), or salt thereof, according to any one of embodiments 10.8 to 10.10, wherein the Suzuki coupling reaction conditions are in the presence of an inorganic base, for example K₃PO₄.

Embodiment 10.12: The method for preparing a compound of formula (III), or salt thereof, according to any one of embodiments 10.8 to 10.10, wherein the compound of formula (V), or salt thereof, is

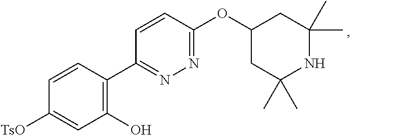 [SMILES: Cc1ccc(S(=O)(=O)Oc2ccc(-c3ccc(OC4CC(C)(C)NC(C)(C)C4)nn3)c(O)c2)cc1]

or a salt thereof.

Embodiment 10.13: A method for preparing a compound of formula (V),

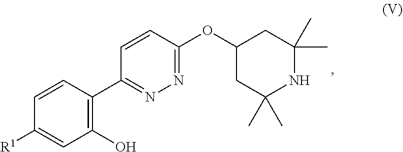

or salt thereof,
wherein R¹ is —OR², and R² is a hydroxyl activating group (e.g. Ts), comprising:
   i) preparing a compound of formula (VI),

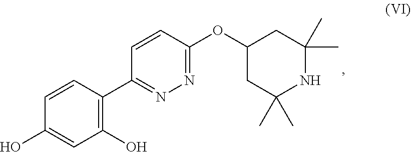 [SMILES: CC1(C)CC(Oc2ccc(-c3ccc(O)cc3O)nn2)CC(C)(C)N1]

or salt thereof,
according to any one of methods of embodiments 10.1 to 10.3; and
   ii) reacting the compound of formula (VI), or salt thereof, according to any one of embodiments 10.4 to 10.7, to obtain the compound of formula (V), or salt thereof.

Embodiment 10.14: A method for preparing a compound of formula (III), or salt thereof,

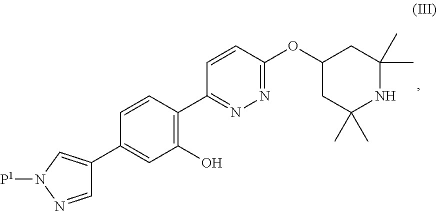

wherein P¹ is a nitrogen protecting group,
comprising:
   i) preparing a compound of formula (V),

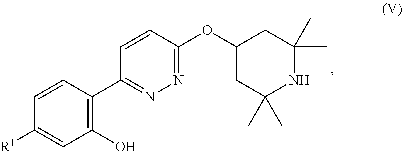

or salt thereof,
wherein R¹ is —OR², and R² is a hydroxyl activating group (e.g Ts),
from the compound of formula (VI),

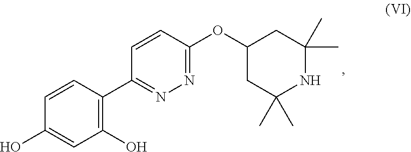 [SMILES: CC1(C)CC(Oc2ccc(-c3ccc(O)cc3O)nn2)CC(C)(C)N1]

or salt thereof,
according to any one of methods of embodiments 10.4 to 10.7; and
   ii) reacting the compound of formula (V), or salt thereof, according to any one of embodiments 10.8 to 10.12, to obtain the compound of formula (III), or salt thereof.

Embodiment 10.15: A method for preparing a compound of formula (III), or salt thereof,

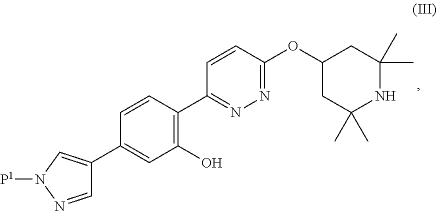

wherein P¹ is a nitrogen protecting group,
according to embodiment 10.14, wherein the compound of formula (VI) or salt thereof is prepared according to any one of methods of embodiments 10.1 to 10.3.

Embodiment 10.16: A compound of formula (V)

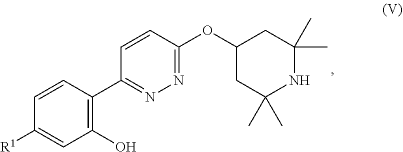

or salt thereof,
wherein R¹ is —OR², and R² is a hydroxyl activating group (e.g Ts).

Embodiment 10.17: The compound of formula (VI),

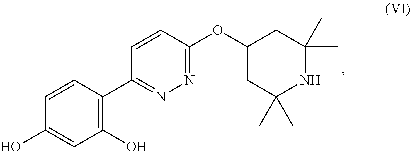 [SMILES: CC1(C)CC(Oc2ccc(-c3ccc(O)cc3O)nn2)CC(C)(C)N1]

or salt thereof.

Embodiment 10.18: A method for preparing a compound of formula (VIII), or salt thereof,

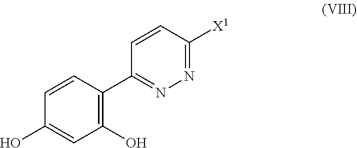

wherein
X¹ is halo;
comprising
reacting a compound of formula (X), or salt thereof,

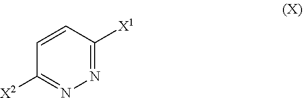

wherein
X¹ is halo;
X² is halo;
with a compound of formula (IX), or salt thereof,

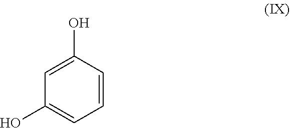

under Friedel-Crafts reaction conditions
to provide the compound of formula (VIII), or salt thereof.

Embodiment 10.19: A compound of formula (III),

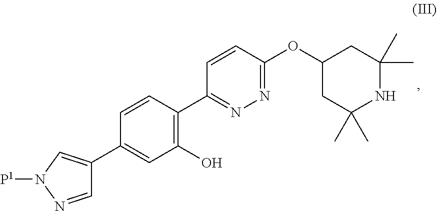

or a salt thereof,
wherein P¹ is a nitrogen protecting group, such as the compound of formula (Ill-1),

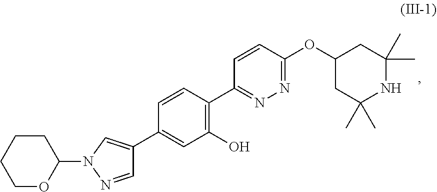 [SMILES: CC1(C)CC(Oc2ccc(-c3ccc(-c4cnn(C5CCCCO5)c4)cc3O)nn2)CC(C)(C)N1]

or a salt thereof.

Embodiment 10.20: A compound of formula (VIII), or salt thereof,

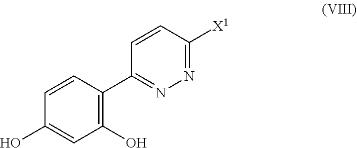

wherein
X¹ is halo (e.g. Cl).

Embodiment 10.21: A method for preparing a compound of formula (VI), or salt thereof,

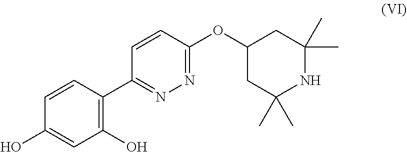 [SMILES: CC1(C)CC(Oc2ccc(-c3ccc(O)cc3O)nn2)CC(C)(C)N1]

comprising
   i) preparing a compound of formula (VIII), or salt thereof,

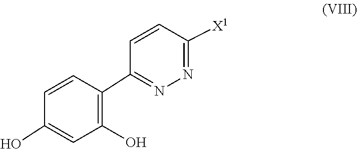

wherein
X¹ is halo,
according to embodiment 10.18; and
ii) reacting the compound of formula (VIII), or salt thereof, according to any one of embodiments 10.1 to 10.3 to obtain the compound of formula (VI), or salt thereof.

Embodiment 10.22: A method for preparing a compound of formula (I), or salt thereof,

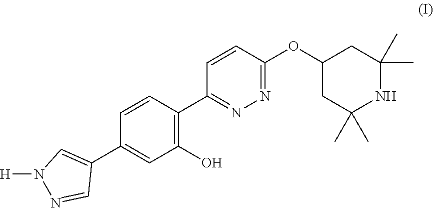 [SMILES: CC1(C)CC(Oc2ccc(-c3ccc(-c4cn[nH]c4)cc3O)nn2)CC(C)(C)N1]

comprising
reacting a compound of formula (III), or salt thereof,

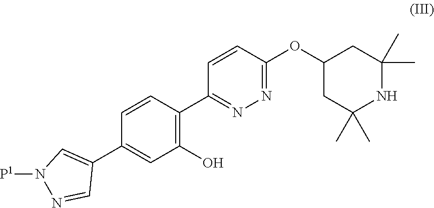

wherein
P¹ is a nitrogen protecting group,
under nitrogen deprotecting conditions to provide the compound of formula (I), or salt thereof.

Embodiment 10.23: A method for preparing a compound of formula (I), or salt thereof,

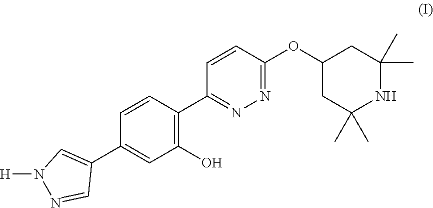 [SMILES: CC1(C)CC(Oc2ccc(-c3ccc(-c4cn[nH]c4)cc3O)nn2)CC(C)(C)N1]

comprising
   i) reacting a compound of formula (V), or salt thereof,

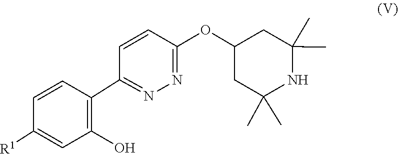

wherein
R¹ is —OR², and R² is a hydroxyl activating group (e.g. Ts),
according to any one of embodiments 10.8 to 10.12,
to provide the compound of formula (III), or salt thereof,

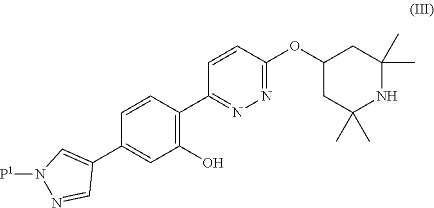

wherein
P¹ is a nitrogen protecting group; and
   ii) reacting the compound of formula (III), or salt thereof, according to embodiment 10.22 to provide the compound of formula (I), or salt thereof.

Embodiment 10.24: A method for preparing a compound of formula (I), or salt thereof,

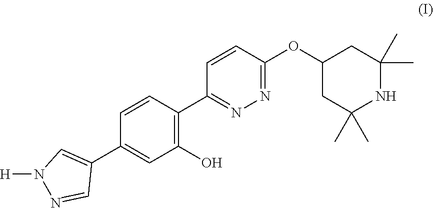 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof,
according to any one of embodiments 10.4 to 10.7, to provide the compound of formula (V), or salt thereof,
   ii) reacting the compound of formula (V), or salt thereof, according to any one of embodiments 10.8 to 10.12 to provide the compound of formula (III), or salt thereof; and
   iii) reacting the compound of formula (III), or salt thereof; according to embodiment 10.22, to provide the compound of formula (I), or salt thereof.

Embodiment 10.25: A method for preparing a compound of formula (V), or salt thereof,

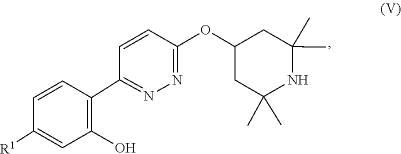

wherein R¹ is —OR², and R² is a hydroxyl activating group (e.g Ts) comprising
   i) reacting the compound of formula (X), or salt thereof, according to embodiment 10.18, to provide the compound of formula (VIII), or salt thereof;
   ii) reacting the compound of formula (VIII), or salt thereof, according to any one of embodiments 10.1 to 10.3 to provide the compound of formula (VI), or salt thereof; and
   iii) reacting the compound of formula (VI), or salt thereof; according to any one of embodiments 10.4 to 10.7, to provide the compound of formula (V), or salt thereof.

Embodiment 10.26: A method for preparing a compound of formula (I), or salt thereof,

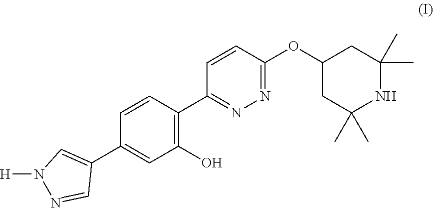 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof, according to any one of embodiments 10.4 to 10.7, to provide the compound of formula (V), or salt thereof,
   ii) reacting the compound of formula (V), or salt thereof, according to any one of embodiments 10.8 to 10.12 to provide the compound of formula (III), or salt thereof; and
   iii) reacting the compound of formula (III), or salt thereof; according to embodiment 10.22, to provide the compound of formula (I), or salt thereof.

Embodiment 10.27: A method for preparing a compound of formula (III), or salt thereof,

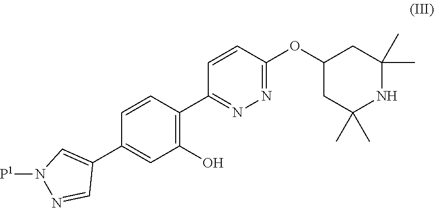

wherein
P¹ is a nitrogen protecting group,
comprising
   i) reacting the compound of formula (X), or salt thereof, according to embodiment 10.18, to provide the compound of formula (VIII), or salt thereof;
   ii) reacting the compound of formula (VIII), or salt thereof, according to any one of embodiments 10.1 to 10.3 to provide the compound of formula (VI), or salt thereof;
   iii) reacting the compound of formula (VI), or salt thereof; according to any one of embodiments 10.4 to 10.7, to provide a compound of formula (V), or salt thereof; and
   iv) reacting the compound of formula (V), or salt thereof; according to any one of embodiments 10.8 to 10.12, to provide the compound of formula (III), or salt thereof.

Embodiment 10.28: A method for preparing a compound of formula (I), or salt thereof,

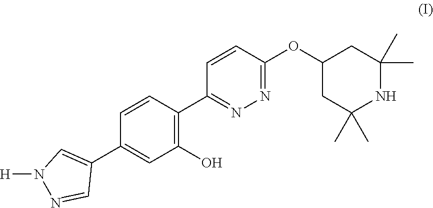 [SMILES: CC1(C)CC(Oc2ccc(-c3ccc(-c4cn[nH]c4)cc3O)nn2)CC(C)(C)N1]

comprising
   i) reacting the compound of formula (X), or salt thereof, according to embodiment 10.18, to provide the compound of formula (VIII), or salt thereof;
   ii) reacting the compound of formula (VIII), or salt thereof, according to any one of embodiments 10.1 to 10.3 to provide the compound of formula (VI), or salt thereof;
   iii) reacting the compound of formula (VI), or salt thereof; according to any one of embodiments 10.4 to 10.7, to provide a compound of formula (V), or salt thereof;
   iv) reacting the compound of formula (V), or salt thereof; according to any one of embodiments 10.8 to 10.12, to provide the compound of formula (III), or salt thereof; and
   v) reacting the compound of formula (III), or salt thereof, according to embodiment 10.22 to provide the compound of formula (I), or salt thereof.

Embodiment 10.29: A method for preparing a compound of formula (I), or salt thereof,

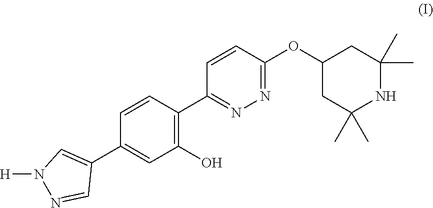 [SMILES: CC1(C)CC(Oc2ccc(-c3ccc(-c4cn[nH]c4)cc3O)nn2)CC(C)(C)N1]

comprising
   i) reacting the compound of formula (VIII), or salt thereof, according to any one of embodiments 10.1 to 10.3 to provide the compound of formula (VI), or salt thereof;
   ii) reacting the compound of formula (VI), or salt thereof; according to any one of embodiments 10.4 to 10.7, to provide a compound of formula (V), or salt thereof;
   iii) reacting the compound of formula (V), or salt thereof; according to any one of embodiments 10.8 to 10.12, to provide the compound of formula (III), or salt thereof; and
   iv) reacting the compound of formula (III), or salt thereof, according to embodiment 10.22 to provide the compound of formula (I), or salt thereof.

Embodiment 10.30: A method for preparing a salt (e.g. a hydrochloride salt) of the compound of formula (I),

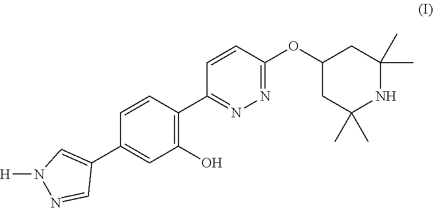 [SMILES: CC1(C)CC(Oc2ccc(-c3ccc(-c4cn[nH]c4)cc3O)nn2)CC(C)(C)N1]

comprising
reacting a compound of formula (III), or salt thereof,

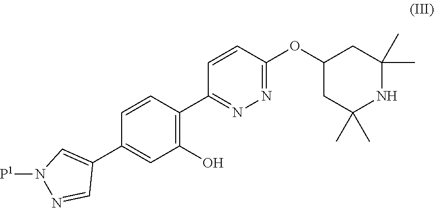

wherein
P¹ is a nitrogen protecting group,
under nitrogen deprotecting conditions (e.g. with HCl) to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.31: A method for preparing a salt (e.g. a hydrochloride salt) of the compound of formula (I),

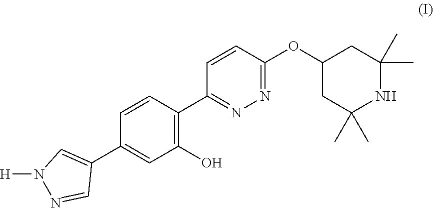 [SMILES: CC1(C)CC(Oc2ccc(-c3ccc(-c4cn[nH]c4)cc3O)nn2)CC(C)(C)N1]

comprising
   i) reacting a compound of formula (V), or salt thereof,

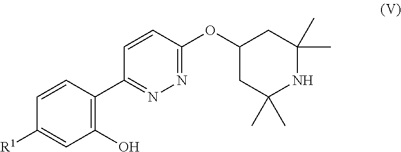

wherein
R¹ is —OR², and R² is a hydroxyl activating group (e.g. Ts),
according to any one of embodiments 10.8 to 10.12,
to provide the compound of formula (III), or salt thereof,

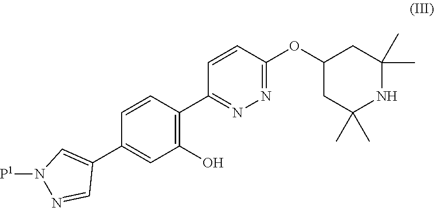

wherein
P¹ is a nitrogen protecting group; and
   ii) reacting the compound of formula (III), or salt thereof, according to embodiment 10.30 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.32: A method for preparing a salt (e.g. a hydrochloride salt) of the compound of formula (I),

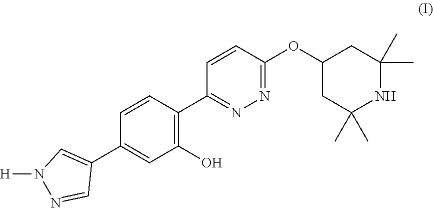 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof,
according to any one of embodiments 10.4 to 10.7, to provide the compound of formula (V), or salt thereof,
   ii) reacting the compound of formula (V), or salt thereof, according to any one of embodiments 10.8 to 10.12 to provide the compound of formula (III), or salt thereof; and
   iii) reacting the compound of formula (III), or salt thereof; according to embodiment 10.30, to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.33: A method for preparing a salt (e.g. a hydrochloride salt) of the compound of formula (I),

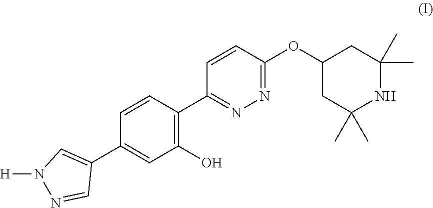 [SMILES: CC1(C)CC(Oc2ccc(-c3ccc(-c4cn[nH]c4)cc3O)nn2)CC(C)(C)N1]

comprising
   i) reacting the compound of formula (VI), or salt thereof, according to any one of embodiments 10.4 to 10.7, to provide the compound of formula (V), or salt thereof,
   ii) reacting the compound of formula (V), or salt thereof, according to any one of embodiments 10.8 to 10.12 to provide the compound of formula (III), or salt thereof; and
   iii) reacting the compound of formula (III), or salt thereof; according to embodiment 10.30, to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.34: A method for preparing a salt (e.g. a hydrochloride salt) of the compound of formula (I),

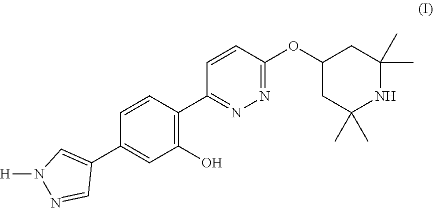 [SMILES: CC1(C)CC(Oc2ccc(-c3ccc(-c4cn[nH]c4)cc3O)nn2)CC(C)(C)N1]

comprising
   i) reacting the compound of formula (X), or salt thereof, according to embodiment 10.18, to provide the compound of formula (VIII), or salt thereof;
   ii) reacting the compound of formula (VIII), or salt thereof, according to any one of embodiments 10.1 to 10.3 to provide the compound of formula (VI), or salt thereof;
   iii) reacting the compound of formula (VI), or salt thereof; according to any one of embodiments 10.4 to 10.7, to provide a compound of formula (V), or salt thereof;
   iv) reacting the compound of formula (V), or salt thereof; according to any one of embodiments 10.8 to 10.12, to provide the compound of formula (III), or salt thereof; and
   v) reacting the compound of formula (III), or salt thereof, according to embodiment 10.30 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.35: A method for preparing a salt (e.g. a hydrochloride salt) of the compound of formula (I),

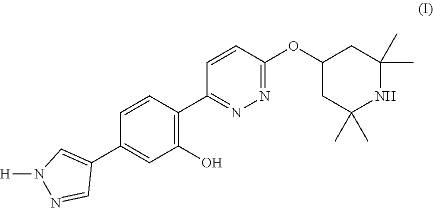 [SMILES: CC1(C)CC(Oc2ccc(-c3ccc(-c4cn[nH]c4)cc3O)nn2)CC(C)(C)N1]

comprising
   i) reacting the compound of formula (VIII), or salt thereof, according to any one of embodiments 10.1 to 10.3 to provide the compound of formula (VI), or salt thereof;
   ii) reacting the compound of formula (VI), or salt thereof; according to any one of embodiments 10.4 to 10.7, to provide a compound of formula (V), or salt thereof;
   iii) reacting the compound of formula (V), or salt thereof; according to any one of embodiments 10.8 to 10.12, to provide the compound of formula (III), or salt thereof; and
   iv) reacting the compound of formula (III), or salt thereof, according to embodiment 10.30 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

Embodiment 10.36: A method for preparing a salt (e.g. a hydrochloride salt) of the compound of formula (I) according to any one of embodiments 10.30 to 10.35, which is optionally followed by crystallization.

Embodiment 10.37: A method for preparing a salt of the compound of formula (I), according to any one of embodiments 10.30 to 10.36, wherein the salt of the compound of formula (I) is a hydrochloride salt {e.g. 5-(1H-Pyrazol-4-yl)-2-{6-[(2,2,6,6-tetramethylpiperidin-4-yl)oxy]pyridazin-3-yl}phenol monohydrochloride monohydrate}.

Embodiment 10.38: A method for preparing a compound of formula (IV)

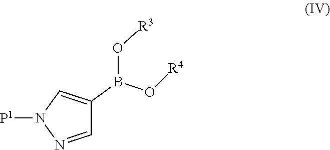

wherein
P¹ is a nitrogen protecting group
R³ is H or C₁₋₄alkyl;
R⁴ is H or C₁₋₄alkyl;
or R³ and R⁴ together to form the group

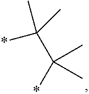 [SMILES: *C(C)(C)C(*)(C)C]

wherein * denotes the point of attachment to each of the oxygen atoms attached to the boron atom,
comprising
   i) reacting a compound of formula (XI), or salt thereof,

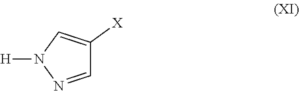

wherein X is halogen (e.g. iodo)
under nitrogen protecting conditions to provide the compound of formula (XII), or salt thereof

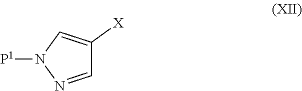

wherein X is halogen (e.g. iodo)
P¹ is a nitrogen protecting group;
   ii) reacting the compound of formula (XII), or salt thereof
      with a compound of formula (XIII),

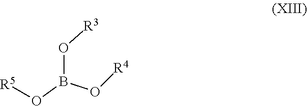

wherein
R³ is H or C₁₋₄alkyl;
R⁴ is H or C₁₋₄alkyl;
R⁵ is C₁₋₄alkyl;
or R³ and R⁴ together to form the group

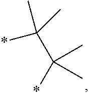 [SMILES: *C(C)(C)C(*)(C)C]

wherein * denotes the point of attachment to each of the oxygen atoms attached to the boron atom,
under Grignard reaction conditions
to provide the compound of formula (IV), or salt thereof.

Embodiment 10.39: A method for preparing a compound of formula (IV), according to embodiment 10.38, wherein the compound of formula (IV) is of formula (IV-1)

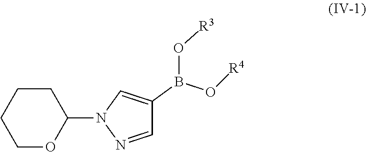

wherein
R³ is H or C₁₋₄alkyl;
R⁴ is H or C₁₋₄alkyl;
or R³ and R⁴ together to form the group

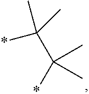 [SMILES: *C(C)(C)C(*)(C)C]

wherein * denotes the point of attachment to each of the oxygen atoms attached to the boron atom.

Embodiment 10.40: A method for preparing a compound of formula (III), according to embodiments 10.8 or 10.9, wherein the compound of formula (IV) or salt thereof is prepared according to the method of embodiments 10.38 or 10.39.

### General Terms

Listed below are definitions of various terms used to describe the present invention. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group.

Alkyl being a radical or part of a radical is a straight or branched (one or, if desired and possible, more times) carbon chain, and is especially C₁-C₇-alkyl, such as C₁-C₄-alkyl, in particular branched C₁-C₄-alkyl, such as isopropyl. The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, iso-propyl, n-butyl, isobutyl, sec-butyl, tert-butyl, in particular methyl, ethyl, n-propyl, iso-propyl, n-butyl, isobutyl, sec-butyl, tert-butyl

The term “ligand” means any compound, achiral or chiral, that can form a complex with a transition metal. Chiral and achiral ligands are in particular those described herein above.

The term “catalyst” means any substance that affects the rate of a chemical reaction by lowering the activation energy for the chemical reaction.

Protecting groups may be present and should protect the functional groups concerned against unwanted secondary reactions, such as acylations, etherifications, esterifications, oxidations, solvolysis, and similar reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

In the present application the term “nitrogen protecting group” generally comprises any group which is capable of reversibly protecting a nitrogen functionality, such as an amino functionality. Suitable nitrogen protecting groups are conventionally used in peptide chemistry and are described e.g. in the relevant chapters of standard reference works such as J. F. W. McOmie, “Protective Groups in Organic Chemistry”, Plenum Press, London and New York 1973; T. W. Greene and P. G. M. Wuts, “Greene's Protective Groups in Organic Synthesis”, Fourth Edition, Wiley, New York 2007; in “The Peptides”; Volume 3 (editors: E. Gross and J. Meienhofer), Academic Press, London and New York 1981, and in “Methoden der organi-schen Chemie” (Methods of Organic Chemistry), Houben Weyl, 4th edition, Volume 15/I, Georg Thieme Verlag, Stuttgart 1974.

As used herein, the term base refers to inorganic bases (e.g. K₂CO₃, ^{t}BuOK, Cs₂CO₃, K₃PO₄, NaOH) or organic bases (e.g. DBU, TMG, NEt₃).

As used herein the term “hydrochloride salt” or “hydrochloride” refers to a salt prepared from the reaction of hydrochloric acid and the compound of interest (e.g. compound of formula (I) or compound of formula (III). Unless explicitly stated, no particular stoichiometry is implied by the use of this term and comprises unsolvated and solvated forms (e.g. hydrates).

As used herein, the term “solvate” is used refers to a molecular complex of a compound of the present invention (including pharmaceutically acceptable salts thereof) with one or more solvent molecules, for example, the hydrochloride salt of the compound of interest (e.g. branaplam) and a stoichiometric amount of one or more pharmaceutically acceptable solvent molecules, for example, water.

As used herein, the term “hydrate” refers to the complex where the solvent molecule is water.

As used herein, the term “crystallization” is used in a broad sense comprising, for example, any process by which dissolved materials precipitate from solution on heterogeneous solid surfaces due to supersaturation, and thus includes reactive crystallization, anti-solvent crystallization and cooling crystallization. In one embodiment it refers to recrystallization.

The term “halogen” or “halo” refers to bromo, chloro, fluoro or iodo, in particular chloro or iodo.

The expression “compound of formula [e.g (VI)] or salt thereof is prepared”, as used in any one of the methods of embodiments or claims described herein, is to be understood as the method further comprising the step(s) of preparing the compound of formula [e.g (VI)] or salt thereof, as indicated.

General Process Conditions

The following, in accordance with the knowledge of a person skilled in the art, applies in general to all processes mentioned hereinbefore and hereinafter, while reaction conditions specifically mentioned above or below are preferred.

All the above-mentioned process steps can be carried out under standard reaction conditions known in the art, unless otherwise specified, preferably those mentioned specifically, in the absence or, customarily, in the presence of solvents or diluents, preferably solvents or diluents that are inert towards the reagents used and dissolve them, in the absence or presence of catalysts, condensation or neutralizing agents (e.g. ion exchangers, such as cation exchangers, e.g. in the H+ form, depending on the nature of the reaction and/or of the reactants), at reduced, normal or elevated temperature, for example in a temperature range of from about −100° C. to about 190° C., preferably from approximately −80° C. to approximately 150° C., for example at from −80 to 60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning. Where required or desired, water-free or absolute solvents can be used.

Where required, the working-up of reaction mixtures, especially in order to isolate desired compounds or intermediates, follows customary procedures and steps, e.g. selected from the group comprising but not limited to extraction, neutralization, crystallization, chromatography, evaporation, drying, filtration, centrifugation and the like.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds described. Special preference is given to reaction conditions that are identical or analogous to those mentioned in the Examples.

As used herein, the terms “free form” or “free forms” refers to the compound in non-salt form, such as the base free form or the acid free form of a respective compound, e.g. the compounds specified herein [e.g. Compound (I)].

As used herein, the terms “salt”, “salts” or “salt form” refers to an acid addition or a base addition salt of a respective compound, e.g. the compounds specified herein. In one embodiment, “salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein may be capable of forming acid and/or base salts. Acid addition salts can be formed with inorganic acids and organic acids:

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Base addition salts can be formed with inorganic and organic bases:

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The following Examples serve to illustrate the invention without limiting the scope thereof, while they on the other hand represent preferred embodiments of the reaction steps, intermediates and/or the process of manufacture of the product in free base form or as a pharmaceutically acceptable salt thereof.

### ABBREVIATIONS

δ chemical shift
br broad
^{t}BuOK potassium tert-butoxide
CyDPEPhos bis(dicyclohexylphosphinophenyl) ether
d doublet
dd doublet of doublet
DMSO-d₆ dimethylsulfoxide deuterated
equiv equivalent
g gram(s)
hr=h hour(s)
HCl hydrochloric acid
¹HNMR proton nuclear magnetic resonance
H₂O water
MS mass spectroscopy
Hz hertz
J coupling constant
Kg=kg kilogram
L litre
m multiplet
MCC microcrystalline cellulose
mg milligram
ml millilitre
mL millilitre
mmol(s) millimole(s)
mol(s) mole(s)
M molarity/molar
MS mass spectrometry
NMR nuclear magnetic resonance
Quadrasil-MP Quadrasil mercaptopropyl
s singlet
t triplet
Tf triflate
tt triplet of triplets
Ts tosylate/tosyl
vol. volume (mL) ratio of solvent over the weight (mg) of the starting material
W weight (g) ratio of the solvent over the weight (g) of starting material
wt % percentage weight
DBU 1,8-Diazabicyclo[5.4. 0]undec-7-ene
TMG 1,1,3,3-Tetramethyiguanidine
LDA Lithium diisapropyarnide
LHMDS Lithium bis(trimethylsilyl)amide

### EXPERIMENTAL

NMR spectra were obtained on a BrukerAvance Ill 400 MHz spectrometer operating at 400 MHz for ¹H and 100 MHz for ¹³C. Chemical shifts (δ) are reported in ppm relative to the tetramethylsilane signal (0 ppm) or residual protio-solvent (2.50 ppm for DMSO) for ¹H-NMR spectra and relative to the solvent resonance (39.5 ppm for DMSO) for ¹³C-NMR spectra. High-resolution mass spectra (electrospray ionization, ESI-TOF) was performed on a Waters Xevo G2-XS QTof mass spectrometer.

### Example 1

### Synthesis of 4-(6-Chloropyridazin-3-yl)benzene-1,3-diol

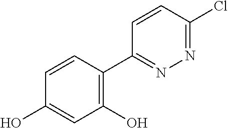 [SMILES: Oc1ccc(-c2ccc(Cl)nn2)c(O)c1]

To a glass-lined 10 L reactor, was added sulfolane (240.0 g) at 30° C., followed by addition of AlCl₃ (69.8 g, 523.6 mmol, 1.30 equiv.) in three portions. The mixture was heated to 85±5° C. to dissolve the solid material and cooled down to 45±5° C., before the addition of 3,6-Dichloropyridazine (60.0 g, 402.7 mmol, 1.0 equiv.) and Benzene-1,3-diol (53.2 g, 483.3 mmol, 1.2 equiv.). The reaction mixture was heated to 75±10° C. for 3 hr, then heated to 93±3° C. and stirred for additional 16 hr. The reaction mixture was then cooled to 50±5° C., and acetonitrile (240.0 g) was added. The solution was further cooled down to 25±5° C. and transferred to a dropping funnel. To a new glass-lined 10 L reaction containing acetonitrile (240.0 g) and water (480.0 g), was added the reaction mixture from the dropping funnel dropwise, and a solid precipitated out. Upon completion of the addition, the suspension was heated to 80±5° C. to obtain a clear solution. The solution was cooled to 63±3° C. over 1 hr, and aged at this temperature for 1 hr, the product crystallized out from the biphasic solution. The suspension was cooled down to 0±5° C. over 5 hr, and aged at this temperature for 2 hr. The solid product was then collected by filtration, and dried in a full vacuum oven (80° C.) over 16 hr, to give the title product as a bright yellow solid.

¹H NMR (400 MHz, DMSO₆) δ 12.05 (br, 1H), 10.05 (s, 1H), 8.36 (d, J=9.3 Hz, 1H), 7.93 (d, J=9.3 Hz, 1H), 7.82 (d, J=8.6 Hz, 1H), 6.42 (dd, J=8.5, 2.5 Hz, 1H), 6.41 (s, 1H);

MS: calc'd m/e for [M+H⁺] C₁₀H₈ClN₂O₂: 223.0, found 223.0.

### Example 2

### Synthesis of 4-{6-[(2,2,6,6-Tetramethylpiperidin-4-yl)oxy]pyridazin-3-yl}benzene-1,3-diol

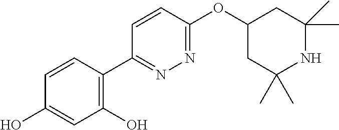 [SMILES: CC1(C)CC(Oc2ccc(-c3ccc(O)cc3O)nn2)CC(C)(C)N1]

To a glass-lined 1 L reaction vessel was added 4-(6-Chloropyridazin-3-yl)benzene-1,3-diol (50.0 g, 224.6 mmol, 1.0 equiv.), 2,2,6,6-Tetramethylpiperidin-4-ol (70.6 g, 449.2 mmol, 2.0 equiv.) and DMSO (300.0 g), sequentially. The mixture was heated to 40±5° C., while stirring. To the mixture, was added ^{t}BuONa (107.9 g, 1122.9 mmol, 5.0 equiv.) in portions. The mixture was heated to 65±5° C. to become a deep red solution and stirred for 16 hr. The solution was cooled to 45±5° C., and quenched with water (900.0 g). The solution was warmed to 65±5° C., and a 31% HCl solution in water (132.1 g, 1122.9 mmol, 5.0 equiv.) was added dropwise at this temperature, and a solid slowly precipitated out. The suspension was cooled down to 20±5° C. over 5 hr, and aged at this temperature for 2 hr. A solid was collected by filtration and dried in a full vacuum oven (80° C.) for 16 hr, to give the title compound as a greyish solid.

¹H NMR (400 MHz, DMSO-_{d6}/MeOD-_{d4}) δ 8.14 (d, J=9.6 Hz, 1H), 7.62 (d, J=8.8 Hz, 1H), 7.16 (d, J=9.5 Hz, 1H), 6.35 (d, J=8.1 Hz, 1H), 6.31 (s, 1H), 5.58 (tt, J=11.3 Hz, 1H), 2.07 (dd, J=12.4, 4.0 Hz, 2H), 1.28 (t, J=8.0 Hz, 2H), 1.22 (s, 6H), 1.11 (s, 6H);

MS: calc'd m/e for [M+H⁺] C₁₉H₂₆N₃O₃: 344.2, found 344.2.

### Example 3

### Synthesis of 3-Hydroxy-4-{6-[(2,2,6,6-tetramethylpiperidin-4-yl)oxy]pyridazin-3-yl}phenyl 4-methylbenzene-1-sulfonate-hydrogen chloride (1/1)

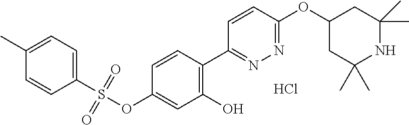

To 0.5 L glass-lined reactor was added 4-{6-[(2,2,6,6-Tetramethylpiperidin-4-yl)oxy]pyridazin-3-yl}benzene-1,3-diol (15 g, 96.05 wt %, 42.0 mmol, 1 equiv.), K₃PO₄ (44.5 g, 209.8 mmol, 5.0 equiv.) and TsCl (9.6 g, 50.3 mmol, 1.2 equiv.), followed by addition of acetonitrile (120 g, 8.0 W). The heterogeneous mixture was warmed to 80±3° C. and stirred for 16 hr. The heterogeneous mixture was cooled to 60±5° C., followed by addition of a diluted HCl solution (54.3 g, 31% HCl in 360 g water, 461.5 mmol, 11 equiv.) over 2 hr, and the product precipitated out from the solution. Upon completion of the addition, the suspension was cooled to 25° C. and the crude product was collected by vacuum filtration. The filter cake was rinsed with 60 g of water, and 60 g of ethanol sequentially. The filter cake was dried in a full vacuum oven at 80° C. for 16 hr, to give the title compound as a greyish solid.

¹H NMR (400 MHz, DMSO-_{d}s/MeOD-_{d4}) δ 9.19 (br, d, J=11.8 Hz, 1H), 8.42 (br, d, J=11.8 Hz, 1H), 8.32 (d, J=9.5 Hz, 1H), 7.90 (d, J=8.8 Hz, 1H), 7.79 (d, J=8.3 Hz, 2H), 7.50 (br, 1H), 7.49 (s, 2H), 7.414 (d, J=9.5 Hz, 1H), 6.70 (d, J=2.4 Hz, 1H), 6.61 (dd, J=8.7, 2.4 Hz, 1H), 5.69 (m, 1H), 2.42 (s, 3H), 2.3 (m, 2H), 1.80 (br, t, J=12.0 Hz, 2H), 1.50 (d, J=8.6 Hz, 12H);

MS: calc'd m/e for [M+H⁺] C₂₆H₃₂N₃O₅S: 534.2 (free base+H⁺=498.2), found 498.2.

### Example 4

### Synthesis of 1-(Oxan-2-yl)-4-(4,4,5,5-tetramethyl-1,3,2-dioxaborolan-2-yl)-1H-pyrazole

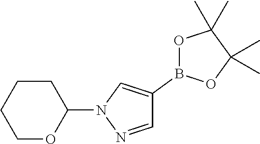 [SMILES: CC1(C)OB(c2cnn(C3CCCCO3)c2)OC1(C)C]

To a 400 mL double-jacketed glass reactor, was added 3,4-dihydro-2H-pyran (10.0 g, 115.5 mmol, 1.13 equiv.) dissolved in tetrahydrofuran (40 g) methanesulfonic acid (0.05 g, 0.51 mmol, 0.005 equiv.) is added and the solution is heated to 40° C. within 20 minutes. A solution of 4-iodo-1H-pyrazole (20.0 g, 1.0 equiv.) dissolved in tetrahydrofuran (20 g) is added over 60 minutes and the solution is aged for 2.5 hr to complete the reaction to 4-iodo-1-(tetrahydro-2H-pyran-2-yl)-1H-pyrazole. Afterwards the solution is cooled to 20° C., diluted with tetrahydrofurane (40 g) and the solution is cooled to −40° C. (±5° C.). A 2.0 M solution of isopropyl magnesiumchloride in tetrahydrofuran (53.8 g, 110.4 mmol, 1.08 equiv.) is added within 1-2 hr and the resulting suspension is stirred for another 30 minutes. To the mixture is added at −40° C. (±5° C.) 2-methoxy-4,4,5,5-tetramethyl-1,3,2-dioxaborolane (20.0 g, 122.8 mmol, 1.2 equiv.) within 1.5 hr and the resulting mixture is stirred at that temperature for another 4 hr to complete the reaction. Acetic acid (7.06 g, 117.6 mmol, 1.15 equiv.) dissolved in tetrahydrofuran (7.06 g) is added at −40° C. (±5° C.) within 30 minutes and the reaction mixture is heated to 25° C. This solution is added within 30 minutes to a biphasic mixture of n-heptane (72 g) and 5% aqueous sodium chloride solution (72 g) and the resulting biphasic mixture is agitated for another 10 minutes. After phase separation the organic phase is extracted with another portion of 5% aqueous sodium chloride solution (72 g). The organic phase is concentrated under vacuum until 70-80 g residue remained. Another two portions of n-heptane (2×80 g) are added and the distillation is repeated twice until 70 g residue remained. To this residue n-heptane (38 g) is added and the solution is heated to 50° C. The solution is cooled down to 35° C. within 30 minutes, seeded (preparation below) with 1-(Oxan-2-yl)-4-(4,4,5,5-tetramethyl-1,3,2-dioxaborolan-2-yl)-1H-pyrazole (57 mg, 0.2 mmol, 0.002 equiv.) and stirred for 3 hr at 35° C. The suspension is cooled to −15° C. within 7 hr and stirred at that temperature for another 7 hr. Afterwards the solid was collected by filtration. Due to its high solubility, the filter cake was not rinsed. It was dried in a full vacuum oven at 40° C. for 16 hr, to give the title product as a white solid.

¹H NMR (400 MHz, DMSO-_{d6}) δ 8.05 (s, 1H), 7.61 (s, 1H), 5.42 (dd, J=10.0, 2.4 Hz, 1H), 3.90 (br dd, J=11.1, 1.4 Hz, 1H), 3.57-3.64 (m, 1H), 2.09 (br d, J=1.3 Hz, 1H), 1.83-1.95 (m, 2H), 1.61-1.68 (m, 1H), 1.48-1.55 (m, 2H), 1.20-1.30 (m, 12H);

MS: calc'd m/e for [M+H⁺] C₁₄H₂₃BN₂O₃: 279.2, found 279.2.

Preparation of Seed Suspension:

1-(Oxan-2-yl)-4-(4,4,5,5-tetramethyl-1,3,2-dioxaborolan-2-yl)-1H-pyrazole (57 mg, 0.2 mmol) was suspended in n-heptane (0.3 mL) in a 1 mL flask at 20° C. The suspension was sonicated for 1 minute in a water bath at 20° C. with ice cooling to keep the temperature constant. This suspension was used for the seeding in the above protocol.

### Example 5

### Synthesis of 5-[1-(Oxan-2-yl)-1H-pyrazol-4-yl]-2-{6-[(2,2,6,6-tetramethylpiperidin-4-yl)oxy]pyridazin-3-yl}phenol

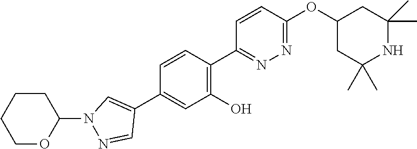 [SMILES: CC1(C)CC(Oc2ccc(-c3ccc(-c4cnn(C5CCCCO5)c4)cc3O)nn2)CC(C)(C)N1]

To 0.5 L glass-lined reactor equipped with an impeller agitator was added 3-Hydroxy-4-{6-[(2,2,6,6-tetramethylpiperidin-4-yl)oxy]pyridazin-3-yl}phenyl 4-methylbenzene-1-sulfonate-hydrogen chloride, 12.85 g, 24.1 mmol, 1 equiv.), K₃PO₄ (15.32 g, 28.9 mmol, 3.0 equiv.) and 1-(Oxan-2-yl)-4-(4,4,5,5-tetramethyl-1,3,2-dioxaborolan-2-yl)-1H-pyrazole (8.03 g, 28.9 mmol, 1.2 equiv.), followed by addition of cyclopentyl methyl ether (103 mL, 8 vol.) and H₂O (26 mL, 2 vol.). The heterogeneous mixture was warmed to 83±5° C., and stirred vigorously under N₂ for 1 hr. To the clear solution was added [Pd(C₃H₅)Cl]₂ (0.176 g, 0.48 mmol, 0.02 equiv.), and CyDPEPhos (0.677 g, 1.20 mmol, 0.05 equiv.). The reaction was stirred at 83±5° C. under N₂ for 16 hr. The reaction mixture was cooled to 40 t 5° C., followed by addition of EtOAc (130 mL, 10 vol.) and H₂O (130 mL, 10 vol.). The mixture was stirred at 40±5° C. for 1 hr, kept still for 0.5 hr, followed by phase separation. The organic layer was passed through a MCC pad, and the filtrate was distilled under vacuo until 45 g residue remained. The residue was warmed to 60±5° C., and n-heptane (240 mL) was added dropwise over 2 hr. The suspension was cooled to 20±5° C. over 2 hr, and aged for 2 hr. The solid was collected by filtration, rinsed with 10 mL 0° C. EtOH. The cake was transferred back to the 0.5 L reactor (pre-cleaned), and Quadrasil-MP (4 g) and toluene (350 mL) were added. The mixture was warmed to 60° C. and stirred for 4 hr. The suspension was cooled to 40 t 5° C. Quadrasil-MP was removed by filtration. The filtrate was distilled under vacuo until 50 g residue remained. The residue was warmed to 95±5° C. to give a clear solution. To the solution was added n-heptane (50 mL) over 1 hr, then cooled to 85±5° C. and aged for 1 hr, while the product crystallized out from the solution. To the suspension, was added n-heptane (103 mL) dropwise over 2 hr, followed by aging at 85±5° C. for 1 hr. The suspension was cooled to 60±5° C. over 4 hr, aged for 1 hr, and then the suspension was cooled to 10±5° C. over 2 hr. The solid was collected by filtration. The cake was rinsed with 10 mL 0° C. EtOH, and dried in a full vacuum oven at 60° C. for 16 hr, to give the tittle product as a yellow solid.

¹H NMR (400 MHz, DMSO-_{d6}) δ 13.24 (br, s, 1H), 8.44 (d, J=9.5 Hz, 2H), 8.03 (s, 1H), 7.94 (d, J=8.3 Hz, 1H), 7.38 (d, J=9.4 Hz, 1H), 7.26 (m, 2H), 5.65 (tt, J=11.1 Hz, 1H), 5.42 (dd, J=10.0, 2.1 Hz, 1H), 3.96 (br, d, J=12.1 Hz, 1H), 3.66 (m, 1H), 2.15 (m, 1H), 2.09 (dd, J=4.0, 8.2 Hz, 2H), 1.95 (m, 2H), 1.70 (br, m, 1H), 1.58 (m, 2H), 1.27 (t, J=12.1 Hz, 2H), 1.24 (s, 6H), 1.11 (s, 6H);

MS: calc'd m/e for [M+H⁺] C₂₇H₃₅N₅O₃: 478.3, found 478.3.

### Example 6

### Synthesis of 5-(1H-Pyrazol-4-yl)-2-{6-[(2,2,6,6-tetramethylpiperidin-4-yl)oxy]pyridazin-3-yl}phenol

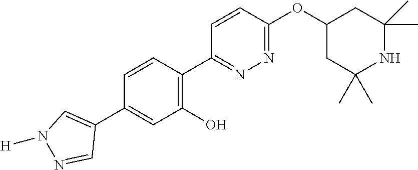 [SMILES: CC1(C)CC(Oc2ccc(-c3ccc(-c4cn[nH]c4)cc3O)nn2)CC(C)(C)N1]

To a 10 L glass-lined reactor, was added 5-[1-(Oxan-2-yl)-1H-pyrazol-4-yl]-2-{6-[(2,2,6,6-tetramethylpiperidin-4-yl)oxy]pyridazin-3-yl}phenol (318 g, 665.8 mmol, 1 equiv.) and methanol (3.8 kg) to give a clear solution. To the solution was added 31% HCl solution in water (852 g, 12 mol, 18 equiv.), followed by stirring at 30° C. for 5 hr. To the solution was added H₂O (3.0 kg), followed by slow addition of a solution of NaOH (133.2 g NaOH in 2.0 L H₂O), while the temperature was maintained under 35° C. The mixture was then cooled to 15° C. over 1 hr, and aged for 16 hr. The solid was collected by filtration, and the filter cake was rinsed with H₂O (2.0 L) and MeOH—H₂O (600 g, 1:1, w/w) sequentially. The wet cake was then dried in a full vacuum oven at 70° C. for 8 hr to give the title compound as a light yellow solid.

¹H NMR (400 MHz, DMSO-_{d6}) δ 13.17 (br, s, 1H), 13.04 (br, s, 1H), 8.45 (d, J=9.7 Hz, 1H), 8.14 (br, s, 2H), 7.93 (d, J=8.3 Hz, 1H), 7.41 (d, J=9.5 Hz, 1H), 7.26 (d, J=1.7 Hz, 1H), 7.24 (dd, J=8.4, 1.7 Hz, 1H), 5.67 (tt, J=11.0 Hz, 1H), 2.17 (br, d, J=9.3 Hz, 2H), 1.44 (br, s, 2H), 1.33 (s, 6H), 1.23 (s, 6H);

MS: calc'd m/e for [M+H⁺] C₂₂H₂₈N₅O₂: 394.2, found 394.2.

### Example 7

### Synthesis of 5-(1H-Pyrazol-4-yl)-2-{6-[(2,2,6,6-tetramethylpiperidin-4-yl)oxy]pyridazin-3-yl}phenol-hydrochloride

To a 250 mL double-jacketed reactor was added 5.1 g of 5-(1H-Pyrazol-4-yl)-2-{6-[(2,2,6,6-tetramethylpiperidin-4-yl)oxy]pyridazin-3-yl}phenol, 100 g of nPrOH, and 100 g of deionized water. The mixture was stirred at 200 rpm, and heated to 30° C. A 37% solution of HCl in water (1.4 g) was added dropwise, and the mixture was heated to 85° C. at 0.5° C./min and a clear solution was observed. The solution was cooled to 60° C. at 1° C./min, at which point the seed slurry was added (see preparation below). The suspension was aged for 1 h at this temperature, and cooled to −10° C. at 0.1° C./min, aged at this temperature for 1 h, and filtered. The cake was washed with a mixture of nPrOH, and deionized water (45 and 5 g respectively). The isolated solid was dried under vacuum (40 mbar) at 30° C. until constant weight to result in the title compound as a white powder.

Preparation of Seed Suspension:

5-(1H-Pyrazol-4-yl)-2-{6-[(2,2,6,6-tetramethylpiperidin-4-yl)oxy]pyridazin-3-yl}phenol (521 mg, 1.32 mmol) was suspended in methanol and water (8 mL and 0.2 mL) in a 50 mL four-necked flask and heated to 60° C. To the suspension, HCl 37% was added (136 mg, 1.31 mmol) and the resulting mixture was stirred for about 5 min, cooled down to room temperature over 30 min. (Crystallization takes place spontaneously at about 40° C., thick grey suspension). The suspension was stirred overnight (16 h) at room temperature, filtered through a glass filter, and washed with methanol (1 mL). The resulting filter cake was dried under vacuum in a drying oven at room temperature for 2 days, then at 50° C. for 16 h to result in the desired titled form. From this solid, 44 mg were taken, suspended into water and used for the seeding in the above protocol.

XRPD Pattern

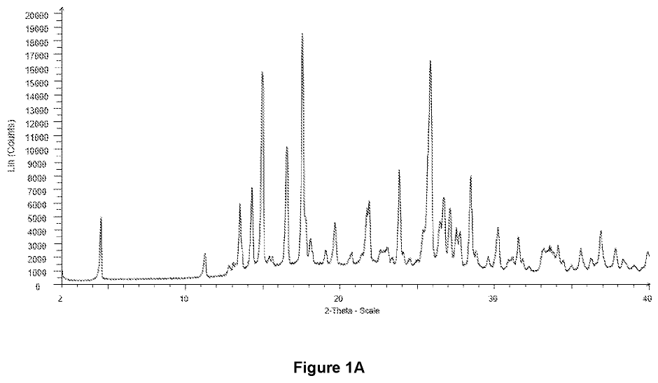

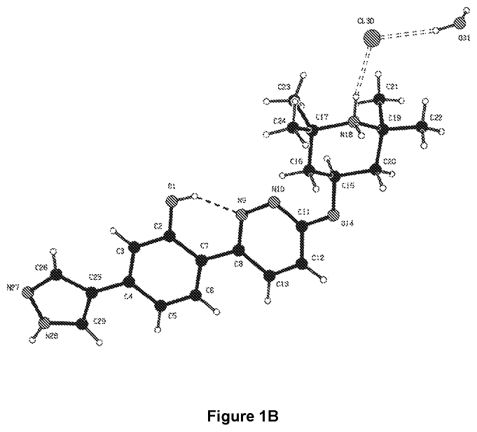

The XRPD pattern of branaplam hydrochloride monohydrate (modification H_{B}), prepared according to the method of this Example 7, is shown in FIG. 1A (previously described in Example 44 in WO2014/028459).

Measurements were performed at a temperature of about 22° C. on an X-Ray powder diffractometer in Bragg-Brentano geometry with a copper X-Ray source of wavelength, A, of 1.5418 Å (CuKα λ=1.5418 Å).

Summary of XRPD Pattern:

This crystalline form is characterized by an XRPD pattern with at least the following peaks at an angle of refraction 2 theta (2θ) of 4.5, 13.8 and 16.6, ±0.2, respectively; preferably characterized by an XRPD pattern with at least the following peaks at an angle of refraction 2 theta (2θ) of 4.5, 11.2, 13.8, 16.6 and 21.9, ±0.2, respectively; more preferably characterized by an XRPD pattern with at least the following peaks at an angle of refraction 2 theta (2θ) of 4.5, 11.2, 13.8, 14.9, 16.6, 21.9, and 28.5, ±0.2, respectively,

In one embodiment, the crystalline Form H_{B} of 5-(1H-Pyrazol-4-yl)-2-{6-[(2,2,6,6-tetramethylpiperidin-4-yl)oxy]pyridazin-3-yl}phenol hydrochloride monohydrate, alternatively named Form H_{B} of 5-(1H-Pyrazol-4-yl)-2-{6-[(2,2,6,6-tetramethylpiperidin-4-yl)oxy]pyridazin-3-yl}phenol monohydrochloride monohydrate (i.e. Form H_{B} of branaplam hydrochloride water 1:1:1) is characterized by an XRPD pattern substantially the same as the XRPD pattern shown in FIG. 1A.

Crystal Structure

The crystal structure (FIG. 1B) of branaplam hydrochloride monohydrate (modification H_{B}), prepared according to the method of this Example 7, was determined using a single crystal of suitable size and quality. A diffractometer equipped with Mo Kα radiation was used. Experimental parameters and relevant information for data treatment is listed herein below:

| Temperature | 100(2) | K |
|---|---|---|
| Wavelength | 1.54178 | Å |
| Crystal system | Monoclinic | |
| Space group | P21/c | |
| Unit cell dimensions | a = 9.831(4) Å a = 90° | |
| b = 7.332 (2) Å | b = 100.86 (9)° | |
| c = 15.721 Å | g = 90° | |
| Volume | 2245.3 (10) | Å3 |
| Z | 4 | |
| Density (calculated) | 1.325 | |
| Absorption coefficient | 1.784 | mm−1 |

| | |
|---|---|
| Final R indices [I > 2sigma(I)] | R1 = 0.0326, wR2 = 0.0811 |
| R indices (all data) | R1 = 0.0307, wR2 = 0.0793 |

### Example 8

### Alternative synthesis of 5-(1H-Pyrazol-4-yl)-2-{6-[(2,2,6,6-tetramethylpiperidin-4-yl)oxy]pyridazin-3-yl}phenol-hydrochloride

To a 400 mL double-jacketed glass reactor, was added 5-[1-(Oxan-2-yl)-1H-pyrazol-4-yl]-2-{6-[(2,2,6,6-tetramethylpiperidin-4-yl)oxy]pyridazin-3-yl}phenol (15 g, 31.4 mmol, 1 equiv.) and ethanol (296 g) to give a suspension. To the suspension was added 37% HCl solution in water (5.57 g, 56.5 mol, 1.8 equiv.). During HCl addition a clear yellow solution is formed. After 15 minutes at 20-23° C. the solution is heated to 40° C. within 30 minutes followed by stirring at 40° C. for 20 hr. During this period a suspension is formed. The suspension is cooled within 2 hr to 14° C. and stirred for another 3 hr at 14° C. The solid was collected by filtration, and the filter cake was rinsed with ethanol (296 g in total) in three portions. The wet cake was dried at 5 mbar in an oven at 40° C. for 16 hr to give a yellow solid. To a 250 mL double-jacketed reactor was added 5.5 g of this solid, 100 g of n-Propanol, and 100 g of deionized water. The mixture was stirred at 200 rpm, and heated to 85° C. at 1.0° C./min and a clear solution was observed. The solution was cooled to 60° C. at 1° C./min, at which point 40 mg of the seed suspended in 500 mg water (preparation of seed suspension as described in Example 7 above) was added. The suspension was aged for 1 h at this temperature, and cooled to −10° C. at 0.1° C./min, aged at this temperature for 1 h, and filtered. The cake was washed with a mixture of n-Propanol, and deionized water (16 and 2 g respectively). The isolated solid was dried under vacuum (20 mbar) at 30° C. until constant weight to result in the title compound as powder. ¹H NMR (400 MHz, DMSO-_{d6}) δ 9.33 (d, J=12.1 Hz, 1H), 8.55 (d, J=12.1 Hz, 1H), 8.48 (d, J=9.6 Hz, 1H), 8.16 (s, 2H), 7.94 (d, J=8.2 Hz, 1H), 7.46 (d, J=9.5 Hz, 1H), 7.28-7.21 (m, 2H), 5.70 (tt, J=10.6, 4.2 Hz, 1H), 2.31 (dd, J=13.2, 4.0 Hz, 2H), 1.90-1.77 (m, 2H), 1.52 (d, J=6.6 Hz, 12H); XRPD pattern as described in Example 7 herein above [i.e. branaplam hydrochloride monohydrate (modification H_{B})].

1. A method for preparing a compound of formula (VI), or salt thereof, 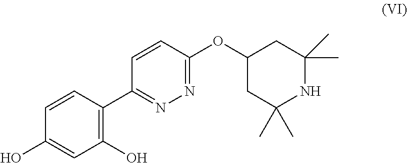 [SMILES: CC1(C)CC(Oc2ccc(-c3ccc(O)cc3O)nn2)CC(C)(C)N1]
comprising
reacting a compound of formula (VII), or salt thereof, 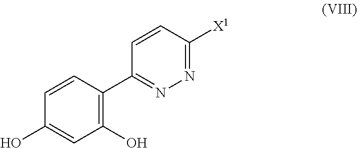
wherein
X¹ is halo;
with a compound of formula (VII), or salt thereof, 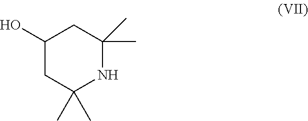
under nucleophilic aromatic substitution (SNAr) reaction conditions,
to provide the compound of formula (VI), or salt thereof.

2. A method for preparing a compound of formula (V), or salt thereof, 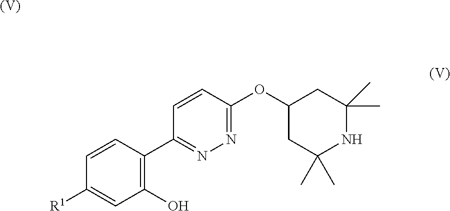
wherein R¹ is —OR², and R² is a hydroxyl activating group (e.g Ts)
comprising
reacting a compound of formula (VI), or salt thereof, 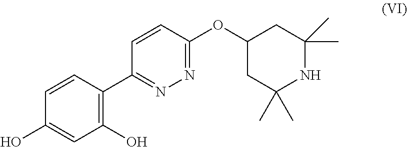 [SMILES: CC1(C)CC(Oc2ccc(-c3ccc(O)cc3O)nn2)CC(C)(C)N1]
under hydroxyl activating reaction conditions
to provide the compound of formula (V), or salt thereof.

3. A method for preparing a compound of formula (III), or salt thereof, 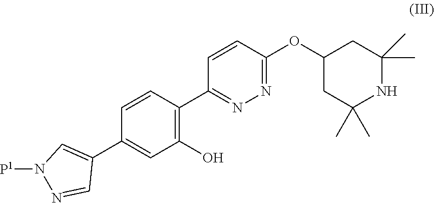
wherein P¹ is a nitrogen protecting group
comprising
reacting a compound of formula (V), or salt thereof, 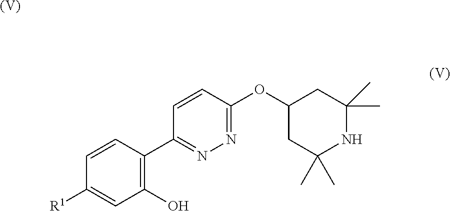
wherein R¹ is —OR², and R² is a hydroxyl activating group (e.g Ts)
with a compound of formula (IV) 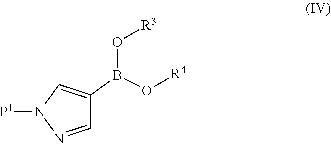
wherein
P¹ is a nitrogen protecting group
R³ is H or C₁₋₄alkyl;
R⁴ is H or C₁₋₄alkyl;
or R³ and R⁴ together to form the group 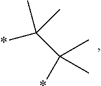 [SMILES: *C(C)(C)C(*)(C)C]
wherein * denotes the point of attachment to each of the oxygen atoms attached to the boron atom,
under Suzuki coupling reaction conditions
to provide the compound of formula (III), or salt thereof.

4. The method for preparing a compound of formula (III), or salt thereof, according to claim 3, further comprising preparing the compound of formula (IV), or salt thereof, according to a method comprising
i) reacting a compound of formula (XI), or salt thereof, 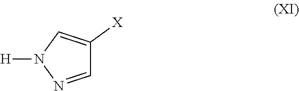
wherein X is halogen (e.g. iodo)
under nitrogen protecting conditions to provide the compound of formula (XII), or salt thereof 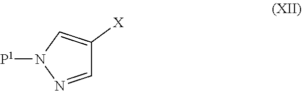
wherein X is halogen (e.g. iodo)
P¹ is a nitrogen protecting group;
ii) reacting the compound of formula (XII), or salt thereof
with a compound of formula (XIII), 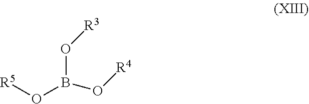
wherein
R³ is H or C₁₋₄alkyl;
R⁴ is H or C₁₋₄alkyl;
R⁵ is C₁₋₄alkyl;
or R³ and R⁴ together to form the group 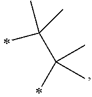
wherein * denotes the point of attachment to each of the oxygen atoms attached to the boron atom,
under Grignard reaction conditions
to provide the compound of formula (IV), or salt thereof.

5. A method for preparing a compound of formula (V), 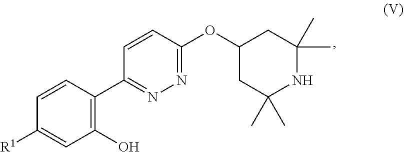
or salt thereof,
wherein R¹ is —OR², and R² is a hydroxyl activating group (e.g. Ts),
comprising:
i) preparing a compound of formula (VI), 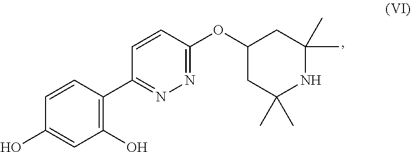 [SMILES: CC1(C)CC(Oc2ccc(-c3ccc(O)cc3O)nn2)CC(C)(C)N1]
or salt thereof,
according to the method of claim 1; and
ii) reacting the compound of formula (VI), or salt thereof, according to claim 2, to obtain the compound of formula (V), or salt thereof.

6. A method for preparing a compound of formula (III), or salt thereof, 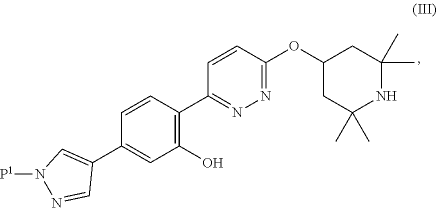
wherein P¹ is a nitrogen protecting group,
comprising:
i) preparing a compound of formula (V), 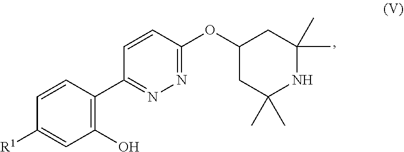
or salt thereof,
wherein R¹ is —OR², and R² is a hydroxyl activating group (e.g Ts),
from the compound of formula (VI), 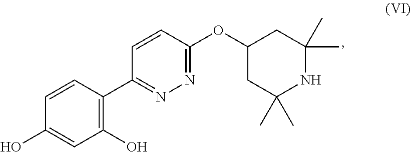 [SMILES: CC1(C)CC(Oc2ccc(-c3ccc(O)cc3O)nn2)CC(C)(C)N1]
or salt thereof,
according to the method of claim 2; and
ii) reacting the compound of formula (V), or salt thereof, according to claims 3 or 4, to obtain the compound of formula (III), or salt thereof.

7. A method for preparing a compound of formula (III), or salt thereof, 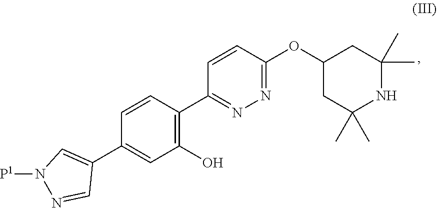
wherein P¹ is a nitrogen protecting group,
according to claim 6, wherein the compound of formula (VI) or salt thereof is prepared according to the method of claim 1.

8. A compound of formula (V) 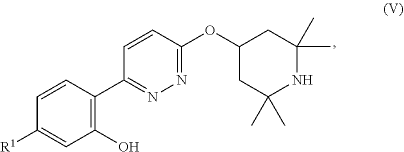
or salt thereof,
wherein R¹ is —OR², and R² is a hydroxyl activating group (e.g Ts).

9. The compound of formula (VI), 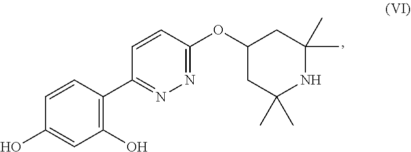 [SMILES: CC1(C)CC(Oc2ccc(-c3ccc(O)cc3O)nn2)CC(C)(C)N1]
or salt thereof.

10. A method for preparing a compound of formula (VIII), or salt thereof, 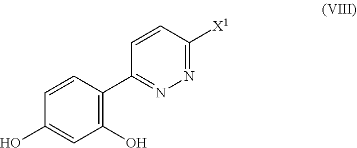
wherein
X¹ is halo;
comprising
reacting a compound of formula (X), or salt thereof, 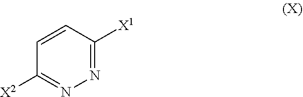
wherein
X¹ is halo;
X² is halo;
with a compound of formula (IX), or salt thereof, 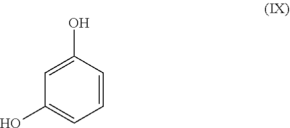
under Friedel-Crafts reaction conditions
to provide the compound of formula (VIII), or salt thereof.

11. A compound of formula (III), 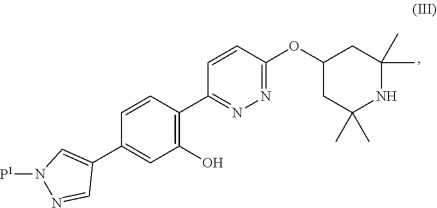
or a salt thereof,
wherein P¹ is a nitrogen protecting group, such as the compound of formula (III-1), 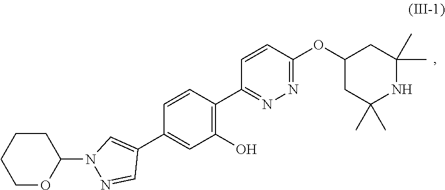 [SMILES: CC1(C)CC(Oc2ccc(-c3ccc(-c4cnn(C5CCCCO5)c4)cc3O)nn2)CC(C)(C)N1]
or a salt thereof.

12. A compound of formula (VIII), or salt thereof, 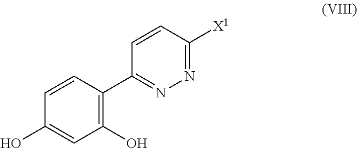
wherein
X¹ is halo (e.g. Cl).

13. A method for preparing a compound of formula (VI), or salt thereof, 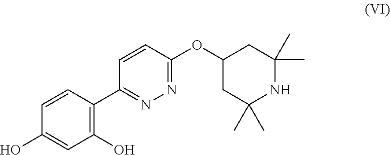 [SMILES: CC1(C)CC(Oc2ccc(-c3ccc(O)cc3O)nn2)CC(C)(C)N1]
comprising
i) preparing a compound of formula (VIII), or salt thereof,
wherein 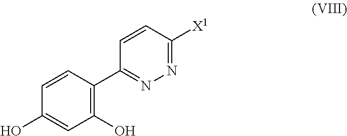
X¹ is halo,
according to claim 10; and
ii) reacting the compound of formula (VIII), or salt thereof, according to claim 1 to obtain the compound of formula (VI), or salt thereof.

14. A method for preparing a compound of formula (I), or salt thereof, 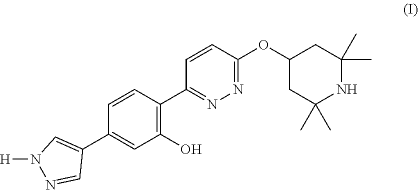 [SMILES: CC1(C)CC(Oc2ccc(-c3ccc(-c4cn[nH]c4)cc3O)nn2)CC(C)(C)N1]
comprising
reacting a compound of formula (III), or salt thereof, 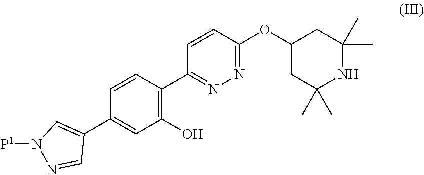
wherein
P¹ is a nitrogen protecting group,
under nitrogen deprotecting conditions to provide the compound of formula (I), or salt thereof.

15. A method for preparing a compound of formula (I), or salt thereof, 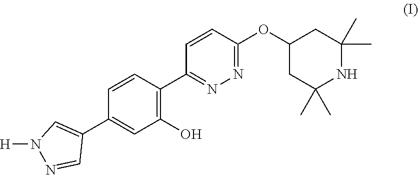 [SMILES: CC1(C)CC(Oc2ccc(-c3ccc(-c4cn[nH]c4)cc3O)nn2)CC(C)(C)N1]
comprising
i) reacting a compound of formula (V), or salt thereof, 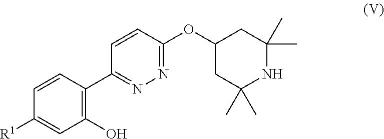
wherein
R¹ is —OR², and R² is a hydroxyl activating group (e.g. Ts),
according to claims 3 or 4,
to provide the compound of formula (III), or salt thereof, 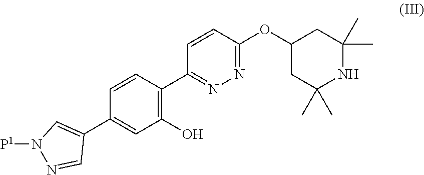
wherein
P¹ is a nitrogen protecting group; and
ii) reacting the compound of formula (III), or salt thereof, according to claim 14 to provide the compound of formula (I), or salt thereof.

16. A method for preparing a compound of formula (I), or salt thereof, 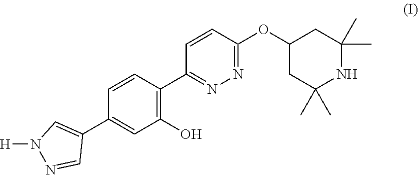 [SMILES: CC1(C)CC(Oc2ccc(-c3ccc(-c4cn[nH]c4)cc3O)nn2)CC(C)(C)N1]
comprising
i) reacting the compound of formula (VI), or salt thereof, according to claim 2, to provide the compound of formula (V), or salt thereof,
ii) reacting the compound of formula (V), or salt thereof, according to claims 3 or 4 to provide the compound of formula (III), or salt thereof; and
iii) reacting the compound of formula (III), or salt thereof; according to claim 14, to provide the compound of formula (I), or salt thereof.

17. A method for preparing a compound of formula (V), or salt thereof, 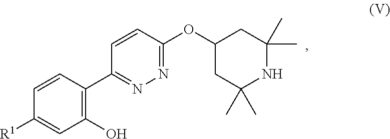
wherein R¹ is —OR², and R² is a hydroxyl activating group (e.g Ts)
comprising
i) reacting the compound of formula (X), or salt thereof, according to claim 10, to provide the compound of formula (VIII), or salt thereof;
ii) reacting the compound of formula (VIII), or salt thereof, according to claim 1 to provide the compound of formula (VI), or salt thereof; and
iii) reacting the compound of formula (VI), or salt thereof; according to claim 2, to provide the compound of formula (V), or salt thereof.

18. A method for preparing a compound of formula (I), or salt thereof, 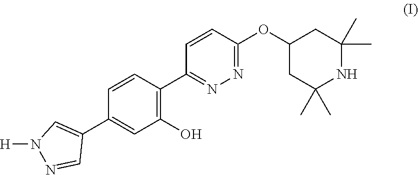 [SMILES: CC1(C)CC(Oc2ccc(-c3ccc(-c4cn[nH]c4)cc3O)nn2)CC(C)(C)N1]
comprising
i) reacting the compound of formula (VI), or salt thereof, according to claim 2, to provide the compound of formula (V), or salt thereof,
ii) reacting the compound of formula (V), or salt thereof, according to claims 3 or 4 to provide the compound of formula (III), or salt thereof; and
iii) reacting the compound of formula (III), or salt thereof; according to claim 14, to provide the compound of formula (I), or salt thereof.

19. A method for preparing a compound of formula (III), or salt thereof, 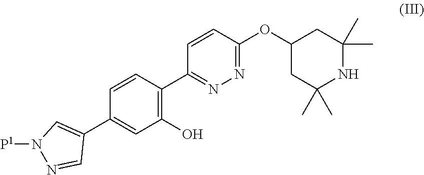
wherein
P¹ is a nitrogen protecting group,
comprising
i) reacting the compound of formula (X), or salt thereof, according to claim 10, to provide the compound of formula (VIII), or salt thereof;
ii) reacting the compound of formula (VIII), or salt thereof, according to claim 1 to provide the compound of formula (VI), or salt thereof;
iii) reacting the compound of formula (VI), or salt thereof; according to claim 2, to provide a compound of formula (V), or salt thereof; and
iv) reacting the compound of formula (V), or salt thereof; according to claims 3 or 4, to provide the compound of formula (III), or salt thereof.

20. A method for preparing a compound of formula (I), or salt thereof, 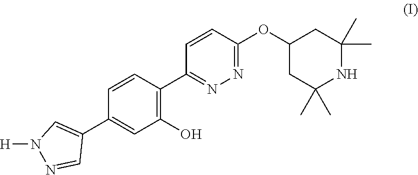 [SMILES: CC1(C)CC(Oc2ccc(-c3ccc(-c4cn[nH]c4)cc3O)nn2)CC(C)(C)N1]
comprising
i) reacting the compound of formula (X), or salt thereof, according to claim 10, to provide the compound of formula (VIII), or salt thereof;
ii) reacting the compound of formula (VIII), or salt thereof, according to claim 1 to provide the compound of formula (VI), or salt thereof;
iii) reacting the compound of formula (VI), or salt thereof; according to claim 2, to provide a compound of formula (V), or salt thereof;
iv) reacting the compound of formula (V), or salt thereof; according to claims 3 or 4, to provide the compound of formula (III), or salt thereof; and
v) reacting the compound of formula (III), or salt thereof, according to claim 14 to provide the compound of formula (I), or salt thereof.

21. A method for preparing a compound of formula (I), or salt thereof, 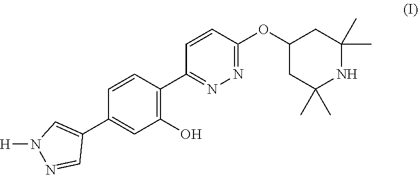 [SMILES: CC1(C)CC(Oc2ccc(-c3ccc(-c4cn[nH]c4)cc3O)nn2)CC(C)(C)N1]
comprising
i) reacting the compound of formula (VIII), or salt thereof, according to claim 1 to provide the compound of formula (VI), or salt thereof;
ii) reacting the compound of formula (VI), or salt thereof; according to claim 2, to provide a compound of formula (V), or salt thereof;
iii) reacting the compound of formula (V), or salt thereof; according to claims 3 or 4, to provide the compound of formula (III), or salt thereof; and
iv) reacting the compound of formula (III), or salt thereof, according to claim 14 to provide the compound of formula (I), or salt thereof.

22. A method for preparing a salt (e.g. a hydrochloride salt) of the compound of formula (I), 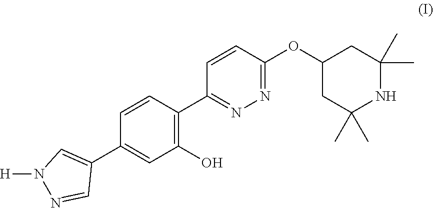 [SMILES: CC1(C)CC(Oc2ccc(-c3ccc(-c4cn[nH]c4)cc3O)nn2)CC(C)(C)N1]
comprising
reacting a compound of formula (III), or salt thereof, 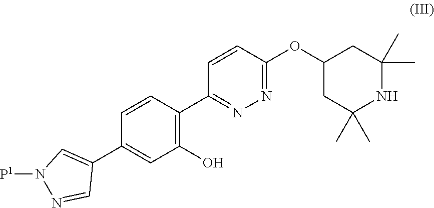
wherein
P¹ is a nitrogen protecting group,
under nitrogen deprotecting conditions (e.g. with HCl) to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

23. A method for preparing a salt (e.g. a hydrochloride salt) of the compound of formula (I), 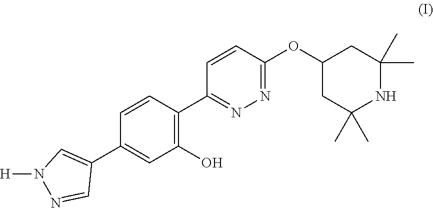 [SMILES: CC1(C)CC(Oc2ccc(-c3ccc(-c4cn[nH]c4)cc3O)nn2)CC(C)(C)N1]
comprising
i) reacting a compound of formula (V), or salt thereof, 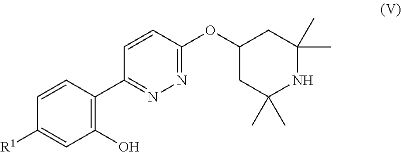
wherein
R¹ is —OR², and R² is a hydroxyl activating group (e.g. Ts),
according to claims 3 or 4,
to provide the compound of formula (III), or salt thereof, 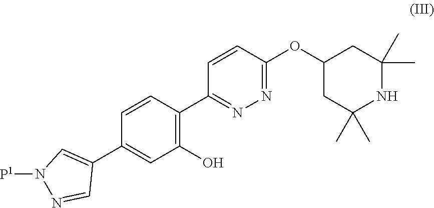
wherein
P¹ is a nitrogen protecting group; and
ii) reacting the compound of formula (III), or salt thereof, according to claim 22 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

24. A method for preparing a salt (e.g. a hydrochloride salt) of the compound of formula (I), 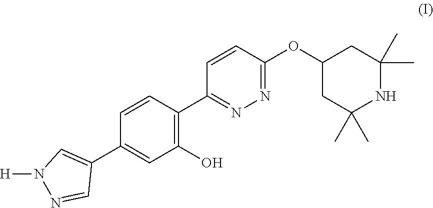 [SMILES: CC1(C)CC(Oc2ccc(-c3ccc(-c4cn[nH]c4)cc3O)nn2)CC(C)(C)N1]
comprising
i) reacting the compound of formula (VI), or salt thereof, according to claim 2, to provide the compound of formula (V), or salt thereof,
ii) reacting the compound of formula (V), or salt thereof, according to claims 3 or 4 to provide the compound of formula (III), or salt thereof; and
iii) reacting the compound of formula (III), or salt thereof; according to claim 22, to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

25. A method for preparing a salt (e.g. a hydrochloride salt) of the compound of formula (I), 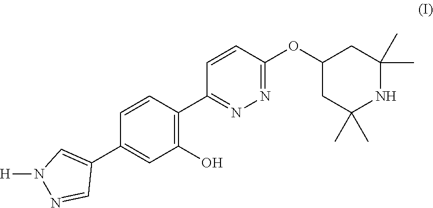 [SMILES: CC1(C)CC(Oc2ccc(-c3ccc(-c4cn[nH]c4)cc3O)nn2)CC(C)(C)N1]
comprising
i) reacting the compound of formula (VI), or salt thereof, according to claim 2, to provide the compound of formula (V), or salt thereof,
ii) reacting the compound of formula (V), or salt thereof, according to claims 3 or 4 to provide the compound of formula (III), or salt thereof; and
iii) reacting the compound of formula (III), or salt thereof; according to claim 22, to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

26. A method for preparing a salt (e.g. a hydrochloride salt) of the compound of formula (I), 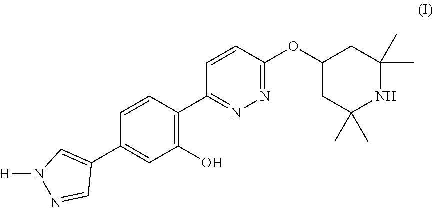 [SMILES: CC1(C)CC(Oc2ccc(-c3ccc(-c4cn[nH]c4)cc3O)nn2)CC(C)(C)N1]
comprising
i) reacting the compound of formula (X), or salt thereof, according to claim 10, to provide the compound of formula (VIII), or salt thereof;
ii) reacting the compound of formula (VIII), or salt thereof, according to claim 1 to provide the compound of formula (VI), or salt thereof;
iii) reacting the compound of formula (VI), or salt thereof; according to claim 2, to provide a compound of formula (V), or salt thereof;
iv) reacting the compound of formula (V), or salt thereof; according to claims 3 or 4, to provide the compound of formula (III), or salt thereof; and
v) reacting the compound of formula (III), or salt thereof, according to claim 22 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

27. A method for preparing a salt (e.g. a hydrochloride salt) of the compound of formula (I), 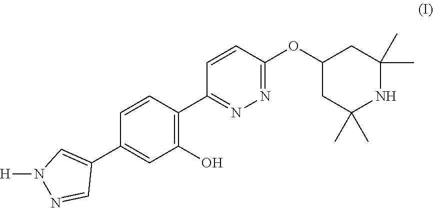 [SMILES: CC1(C)CC(Oc2ccc(-c3ccc(-c4cn[nH]c4)cc3O)nn2)CC(C)(C)N1]
comprising
i) reacting the compound of formula (VIII), or salt thereof, according to claim 1 to provide the compound of formula (VI), or salt thereof;
ii) reacting the compound of formula (VI), or salt thereof; according to claim 2, to provide a compound of formula (V), or salt thereof;
iii) reacting the compound of formula (V), or salt thereof; according to claims 3 or 4, to provide the compound of formula (III), or salt thereof; and
iv) reacting the compound of formula (III), or salt thereof, according to claim 22 to provide a salt (e.g. a hydrochloride salt) of the compound of formula (I) [e.g. 5-(1H-Pyrazol-4-yl)-2-{6-[(2,2,6,6-tetramethylpiperidin-4-yl)oxy]pyridazin-3-yl}phenol monohydrochloride monohydrate].

28. A method for preparing a compound of formula (IV) 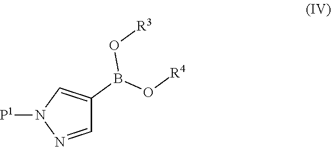
wherein
P¹ is a nitrogen protecting group
R³ is H or C₁₋₄alkyl;
R⁴ is H or C₁₋₄alkyl;
or R³ and R⁴ together to form the group 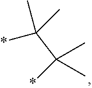 [SMILES: *C(C)(C)C(*)(C)C]
wherein * denotes the point of attachment to each of the oxygen atoms attached to the boron atom,
comprising
i) reacting a compound of formula (XI), or salt thereof, 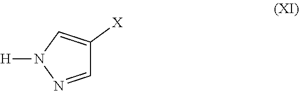
wherein X is halogen (e.g. iodo)
under nitrogen protecting conditions to provide the compound of formula (XII), or salt thereof 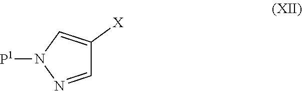
wherein X is halogen (e.g. iodo)
P¹ is a nitrogen protecting group;
ii) reacting the compound of formula (XII), or salt thereof with a compound of formula (XIII), 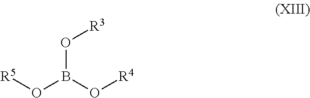
wherein
R³ is H or C₁₋₄alkyl;
R⁴ is H or C₁₋₄alkyl;
R⁵ is C₁₋₄alkyl;
or R³ and R⁴ together to form the group 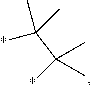 [SMILES: *C(C)(C)C(*)(C)C]
wherein * denotes the point of attachment to each of the oxygen atoms attached to the boron atom,
under Grignard reaction conditions
to provide the compound of formula (IV), or salt thereof.
